(19) United States
(12) Patent Application Publication
(10) Pub. No.: US 2009/0325315 A1
(43) Pub. Date: Dec. 31, 2009

(54) DETECTION METHOD OF TARGET SUBSTANCE, DETECTION REAGENT USED FOR THE SAME, AND THE USES THEREOF

(75) Inventors: Kaoru HIRAI, Kyoto-shi (JP); Yasunori SHIRAKI, Kyoto-shi (JP)

Correspondence Address: HAMRE, SCHUMANN, MUELLER & LARSON, P.C., P.O. BOX 2902, MINNEAPOLIS, MN, 55402-0902, US

(73) Assignee: ARKRAY, INC., Kyoto-shi (JP)

(21) Appl. No.: 12/493,895

(22) Filed: Jun. 29, 2009

(30) Foreign Application Priority Data
Jun. 30, 2008 (JP) 2008-170327

(51) Int. Cl. G01N 33/566; G01N 21/00

(52) U.S. Cl. 436/501; 422/82.05

(57) ABSTRACT

A new detection method, for detecting a target substance using formation of an aggregate due to binding of a target substance and a binding substance that binds thereto, that is excellent in detection accuracy, and sensitivity, and a new detection reagent used for the same are provided. Modifying substances having a maximum diameter of about 50 nm or less bind to a binding substance that binds to a target substance, and a modified binding substance is prepared as a binding reagent. A target substance in a sample is detected by bringing this modified binding reagent into contact with the sample, and optically detecting an aggregate that is formed by binding of the modified binding substance and the target substance in the sample. Preferably, the modifying substance includes biotin or a biotin derivative and further includes avidin or an avidin derivative, and the avidin or the avidin derivative binds to the biotin or the biotin derivative. Further, preferably, the biotin or the biotin derivative binds to the binding substance via a spacer.

### BACKGROUND OF THE INVENTION

1. Field of the Invention

The present invention relates to a detection method of a target substance, a detection reagent used for the same, and the uses thereof.

2. Description of Related Art

As a method of detecting a target substance, a method using a binding substance which specifically bonds to the target substance is widely adopted. In this method, detection of the target substance is performed indirectly by reacting the target substance with the binding substance, confirming whether the target substance and the binding substance are bound, and measuring an amount of the binding of the target substance and the binding substance. Above all, a detection method using an antigen-antibody reaction generally is used. Typical examples thereof include a turbidimetric immunoassay (TIA) and a Latex agglutination-turbidimetric immunoassay (LA) (JP2001133454 A and JP2007315883 A). In the former TIA method, an antibody against the target substance is added to a sample, a complex is formed by an antigen-antibody reaction of a target substance in a sample and the antibody, an aggregate thereof is irradiated with light, and an absorbance is measured. Since the absorbance is correlated with an amount of the target substance in the sample, the amount of the target substance in the sample indirectly can be measured therefrom. In the latter LA method, an antibody that is immobilized to a latex particle is used, a complex is formed by an antigen-antibody reaction of a target substance and the antibody as well as the latex particle to which the antibody is immobilized is aggregated, and this aggregate is detected.

However, these methods have the following problems. That is, with respect to the former TIA method, since the obtained aggregate is small, the measurement sensitivity is insufficient. Particularly, in a case where a concentration of a target substance in a sample is low, it is very difficult to detect. In contrast, with respect to the latter LA method, since the obtained aggregate is large, the measurement sensitivity in a low concentration range is excellent. However, at the same time, a hook phenomenon (also referred to as “a zone phenomenon” such as a prozone phenomenon and a postzone phenomenon), in which a measurement value is decreased, may occur in a high concentration range. The problem of hook phenomenon may be solved, for example, by respectively immobilizing antibodies to plural latex particles of various sizes, performing measurement using each immobilized antibody, and adopting results in which prozone is not occur. However, such method requires plural immobilized antibodies and it takes time and cost. In order to solve the problem of prozone, besides the method described above, there is a method of remeasurement by optimizing dilution ratio by increasing dilution ratio of a sample, for example. However, such method takes time for optimizing dilution and dilution ratio and remeasuing. Further, the LA method has problems of increase in background and decrease in S/N.

Further, in recent years, a disposable small analysis tool is focused, in which a channel, a reaction portion, and the like are miniaturized, such as a microchip and a micro TAS (micro total analysis system: μTAS). In view of the practical use of these small analysis tools and market demand for a point of care examination, it is required to perform detection in a target concentration range without diluting a sample. In this case, a high concentration range should be detected sufficiently without using a diluting means as well as a low concentration range should be detected sufficiently. An example of a method of avoiding the hook phenomenon with respect to an undiluted sample includes a method in which sufficient amount of binding-substance (for example, antibody) to a target substance (for example, antigen) is added, i.e., addition ratio of an antibody relative to an antigen (hereinafter, also referred to as “an antigen-antibody ratio”) is set at a higher level. However, an increase in an antibody amount raises cost. In addition, there is a limitation in performance. In a case of the LA method, it is difficult to avoid the hook phenomenon even when the antigen-antibody ratio is set at higher level. In a case of the TIA method, similar to the case described above, the measurement accuracy in a low concentration range is insufficient.

Further, latex used for the LA method is adsorbed easily by a measurement cell. Therefore, when measurement of a reaction solution of the LA method is performed by sequentially dispensing to a measurement cell of an autoanalyzer, the blank gradually may be deteriorated due to adsorption of the latex to the cell. In order to solve such problem, for example, there is a method for frequently performing alkali washing or the like of the measurement cell. However, it takes time and cost for maintenance. Further, with respect to the microchip and the micro TAS, since a channel, a reaction portion, and the like are miniaturized, for example, reduction of a sample, a reagent, a reaction time, waste, and the like are expected. In a case where the LA method is applied to such analysis tool, normally, an antibody immobilized to a latex particle is placed in a reagent portion of an analysis tool, and stored therein until it is used. However, there is a problem that the latex particle may be adsorbed by the reagent portion during storage. Therefore, in a case of practical use, an amount of antibody that can react to a target substance in a liquid sample supplied to the analysis tool is decreased. Further, in a case where the reagent portion also serves as a measurement portion, the blank value may be increased due to adsorption, and S/N ratio may be deteriorated.

Further, in a case where the LA method and the TIA method are applied to a small analysis tool such as the microchip or the like described above, the following problem may occur. With respect to the small analysis tool, the cell length of a detection portion (a light irradiating portion) that performs an optical detection is a micrometer order and is generally about 100 μm, for example. In contrast, with respect to a quartz cell or the like that conventionally is used for an optical detection, it is known that the cell length thereof is normally 1 cm. However, even when a specimen can be detected with the cell length of 1 cm, if the same specimen is detected with the cell length of about 1/100 (100 μm), sufficient detection sensitivity cannot be obtained from a low concentration range to a high concentration range.

### SUMMARY OF THE INVENTION

Hence, the present invention is intended to provide a new detection method, for detecting a target substance using formation of an aggregate due to binding of the target substance and a binding substance that binds thereto, that is excellent in detection accuracy and sensitivity, and a new detection reagent used for the same.

A detection method of the present invention is a method of detecting a target substance in a sample using a binding substance that binds to the target substance, comprising the following (A) and (B):
(A) bringing the binding substance into contact with the sample, and forming an aggregate by binding of the binding substance and the target substance in the sample; and
(B) detecting the aggregate, wherein
   the binding substance is a binding substance to which modifying substances having the maximum diameter of about 50 nm or less bind.

A detection reagent of the present invention is a detection reagent used for a detection method of a target substance of the present invention, comprising:
a binding substance which binds to the target substance, wherein
the binding substance is a binding substance to which modifying substances having the maximum diameter of about 50 nm or less bind.

A detection tool of the present invention is a detection tool used for a detection method of a target substance of the present invention, comprising: a main body and a detection reagent of the present invention, wherein the detection reagent is placed in the main body.

According to the present invention, a target substance can be detected with excellent accuracy and sensitivity by using a binding substance in which the aforementioned modifying substances are bound. Therefore, the present invention is very useful in an analysis and clinical field.

### BRIEF DESCRIPTION OF THE DRAWINGS

FIG. 1 shows the results of CRP measurement using an avidin-biotinylated antibody in Example 1 of the present invention, FIG. 1A is a graph showing a relationship between a CRP concentration and an absorbance, and FIG. 1B is a graph in which a CRP low concentration range is enlarged.

FIG. 2 shows the results of CRP measurement using an avidin-biotinylated antibody in Example 2 of the present invention, and is a graph showing a relationship between a CRP concentration and an absorbance.

FIG. 3 shows the results of CRP measurement using a streptavidin-biotinylated antibody in Example 3 of the present invention, and is a graph showing a relationship between a CRP concentration and an absorbance.

FIG. 4 shows the results of CRP measurement using a biotinylated antibody in Example 4 of the present invention, FIG. 4A is a graph showing a relationship between a CRP concentration and an absorbance, and FIG. 4B is a graph in which a CRP low concentration range is enlarged.

FIG. 5 shows the results of CRP measurement using an avidin-biotinylated antibody in Example 6 of the present invention, FIG. 5A is a graph showing a relationship between a logarithmic displayed CRP concentration and an absorbance calculated from a transmitted light intensity as well as a relationship between a logarithmic displayed CRP concentration and a scattered light intensity, FIG. 5B is a graph showing a relationship between a constant displayed CRP concentration and the absorbance as well as a constant displayed CRP concentration and the scattered light intensity, and FIG. 5C is a graph showing a relationship between the constant displayed CRP concentration and the absorbance as well as the constant displayed CRP concentration and the scattered light intensity.

FIG. 6 shows the results of CRP measurement by a TIA method using an anti-CRP antibody in Comparative Example 1, FIG. 6A is a graph showing a relationship between a CRP concentration and an absorbance, and FIG. 6B is a graph in which a CRP low concentration range is enlarged.

FIG. 7 shows the results of CRP measurement by a LA method using a latex-bound anti-CRP antibody in Comparative Example 2, FIG. 7A is a graph showing a relationship between a CRP concentration and an absorbance, and FIG. 7B is a graph in which a CRP low concentration range is enlarged.

FIG. 8 is a perspective view showing a scattered light measurement device in an Embodiment of the present invention.

FIG. 9 is a cross-sectional view of the scattered light measurement device shown in FIG. 8.

FIG. 10 is a perspective view showing a scattered light measurement device in another Embodiment of the present invention.

FIG. 11 is a schematic view showing a sample holding tool in another Embodiment of the present invention, FIG. 11A is a perspective view separately showing components, FIG. 11B is a perspective view viewed from the upper side, and FIG. 11C is a perspective view viewed from the back side.

FIG. 12 is a schematic view showing a sample holding tool in yet another Embodiment of the present invention, FIG. 12A is a perspective view separately showing components, FIG. 12B is a perspective view viewed from the upper side, and FIG. 12C is a perspective view viewed from the back side.

FIG. 13 is a perspective view showing a sample holding tool in Example 6 of the present invention.

### DETAILED DESCRIPTION OF THE INVENTION

### <Detection Reagent>

The detection reagent of the present invention is a detection reagent used for a detection method of a target substance of the present invention. The reagent comprises a binding substance that binds to the target substance, and the binding substance is a binding substance to which modifying substances having the maximum diameter of about 50 nm or less bind.

In the present invention, the binding substance is applicable as long as the modifying substances are bound therein, and other aspects thereof are not limited at all. Hereinafter, the binding substance to which the modifying substances bind also is referred to as “a modified binding substance”. The binding form of the modifying substance to the binding substance is not particularly limited. For example, it is preferable that the modifying substance is not dissociated from the binding substance at the time of detecting the target substance, and for example, binding in an irreversible manner is preferable.

For example, the upper limit of the maximum diameter of the modifying substance is preferably about 50 nm or less, more preferably about 30 nm or less, and particularly preferably about 15 nm or less. Further, for example, the lower limit thereof is preferably about 2 nm or more, more preferably about 3 nm or more, and particularly preferably about 5 nm or more. Further, for example, the maximum diameter is preferably in the range of about 2 to 50 nm, more preferably in the range of about 3 to 30 nm, and particularly preferably in the range of about 5 to 15 nm.

The modifying substance is not particularly limited as long as it is in the aforementioned size. Preferably, the modifying substance contains biotin, a biotin derivative, a nucleic acid, a carbon nanotube, and the like. The modifying substance may be formed by one of them or two or more of them. Further, the modifying substance may contain another substance. An example of the other substance includes a spacer, and biotin and the like may be bound to the binding substance via the spacer.

Hereinafter, the biotin and the biotin derivative are referred to as “biotins”. In a case where the modifying substance contains the biotins, hereinafter, the modified binding substance in which the modifying substance is bound is also referred to as “biotinylated binding substance”.

In a case where the modifying substance contains the biotins, another substance further may be bound to the biotins. An example of the other substance includes a spacer described above. Further, it is preferable that the modifying substance contains at least one of avidin and an avidin derivative as the other substance. Hereinafter, the avidin and the avidin derivative are also referred to as “avidins”. In a case where the modifying substance further contains the avidins, the avidins preferably are bound to the biotins. In a case where the modifying substance contains the biotins and the avidins bound thereto, hereinafter, the modified binding substance to which the modifying substances are bound also is referred to as, among the biotinylated binding substances, “avidin-biotinylated binding substance”, “avidin-biotin complex binding substance” or “complex binding substance”.

The binding reaction between biotins and avidins is normally irreversible. Therefore, in the present invention, an avidin-biotinylated binding substance is more preferable since it is excellent in stability as a reagent. Hereinafter, the biotinylated binding substance includes the meaning of “avidin-biotinylated binding substance” unless otherwise described.

With respect to the avidin-biotinylated binding substance, the molar ratio between biotins and avidins is not particularly limited. Since avidins include four subunits, avidins can bind to up to four molecules of biotins per molecule. The molar ratio between biotins and avidins is, for example, about 1:3, preferably about 1:2, and more preferably about 1:1.

For example, as shown below, with respect to the biotinylated binding substance, the biotins preferably are bound to the binding substance against a target substance via a spacer (X). Further, in a case where the biotinylated binding substance contains biotins and avidins, as shown below, it is preferable that the biotins are bound to the binding substance via the spacer (X), and the avidins are bound to the biotins. Preferably, one end of the spacer (X) is bound to a carboxyl group of the biotins, and the binding thereof is preferably an amide binding (—NH—CO—). Further, in a case where the binding substance is, for example, an amine compound such as protein or the like, the other end of the spacer preferably is bound to an amino group of the binding substance, and the binding thereof is preferably an amide binding (—NH—CO—).

Binding substance-X-biotins

Binding substance-X-biotins-avidins

It is preferable that the sum of the length of a condensed heterocycle of biotins and a spacer is, for example, about 10 to 50 Å (about 1 to 5 nm), although the length of the spacer is not particularly limited.

Specific examples of the configuration of the spacer (—X—) are as follows. In the following, with respect to each spacer, the sum of the length of biotins and a spacer is described. Among them, the spacers of (2) and (3) are preferable, and the spacer of (2) is more preferable.

TABLE 1

| -X- | Length of -X-biotins |
|---|---|
| (1) —CO—(CH₂)₅—NH— | 22.4 Å (2.24 nm) |
| (2) —CO—(CH₂)₅—NH—CO—(CH₂)₅—NH— | 30.5 Å (3.05 nm) |
| (3) —CO—(CH₂)₂—CO—NH—(CH₂)₃—O—(CH₂)₂—O—(CH₂)₂—O—(CH₂)₃—NH— | 32.6 Å (3.26 nm) |
| (4) —CO—(CH₂)₂—S—S—(CH₂)₂—NH— | 24.3 Å (2.43 nm) |
| (5) —CO—(CH₂)₂—O—(CH₂)₂—O—(CH₂)₂—O—(CH₂)₂—O—(CH₂)₂—NH— | 29.0 Å (2.90 nm) |

In the biotinylated binding substance, binding of the spacer to the binding substance is not limited to the aforementioned amide binding. The spacer may bind to a thiol group, a carboxyl group, or the like of the binding substance. Examples of such spacers are as follows, although the present invention is not limited thereto.

TABLE 2

| -X- | Length of -X-biotins |
|---|---|
| (6) —C₄H₃NO₂—(CH₂)₂—CO—NH—(CH₂)₂—O—(CH₂)₂—O—(CH₂)₂—NH— | 29.1 Å (2.91 nm) |
| (7) —CH₂—CO—NH—(CH₂)₂—O—(CH₂)₂—O—(CH₂)₂—NH— | 24.7 Å (2.47 nm) |
| (8) —NH—(CH₂)₂—O—(CH₂)₂—O—(CH₂)₂—NH— | 20.4 Å (2.04 nm) |
| (9) —NH—(CH₂)₂—O—(CH₂)₂—O—(CH₂)₂—O—(CH₂)₂—NH— | 22.9 Å (2.29 nm) |
| (10) —NH—(CH₂)₅—NH— | 18.9 Å (1.89 nm) |
| (11) —O—(CH₂)₃—CO—NH—(CH₂)₂—O—(CH₂)₂—O—(CH₂)₂—O—(CH₂)₂—NH— | 36.9 Å (3.69 nm) |
| (12) —NH—(CH₂)₂—O—(CH₂)₂—O—(CH₂)₂—O—(CH₂)₂—O—(CH₂)₂—O—(CH₂)₂—NH— | 43.4 Å (4.34 nm) |

Biotin and biotin derivative are not particularly limited, and examples thereof include those which can bind to avidin or avidin derivative which will be described later. Examples of the biotin include a compound, (5-[(3aS,4S,6aR)-2-oxohexahydro-1H-thieno[3,4-d]imidazol-4-yl]pentanoic acid, represented by the following Formula, a tautomer or a stereoisomer thereof, or their salts.

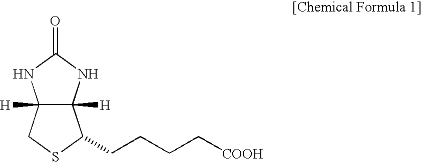

In a case of a salt, examples of the counterion include sodium ion (Na⁺), potassium ion (K⁺), and the like, although it is not particularly limited.

Further, the biotin derivative is not particularly limited and an arbitrary atom may be substituted in the aforementioned biotin. For example, a hydrogen atom may be substituted by halogen such as chlorine, bromine, iodine, fluorine, and the like; or the like.

The avidins are not particularly limited and examples thereof include avidin and an avidin derivative. Examples of the avidin derivative include streptavidin, modified streptavidin, deglycosylated avidin (for example, NeutrAvidin (trade mark), manufactured by Pierce Biotechnology, Inc.), and the like. Examples of the modified streptavidin include streptavidin modified with a hydrazide residue, and the like. Among them, avidin is preferable. Avidins to be bound to the biotins may be derived from natural products or may be synthetic.

Further, the modifying substance may contain avidins instead of biotins. In a case where the modifying substance contains avidins and biotins, the biotins may bind to the binding substance via the avidins.

As described above, the modifying substance may contain a nucleic acid, for example. The modifying substance may be composed of a nucleic acid only, or further may contain another substance. Examples of the other substance include a spacer, and the like. A method of binding the nucleic acid to the binding substance is not particularly limited and conventionally known methods can be adopted. The method can be decided suitably according to types of the binding substance.

The nucleic acid is not particularly limited and examples thereof include polynucleotide and modified polynucleotide. Structural unit of the polynucleotide is not particularly limited and examples thereof include ribonucleotide and deoxyribonucleotide. The polynucleotide may be composed of any one of ribonucleotide and deoxyribonucleotide or may be composed of both of them. Further, the nucleic acid may be an artificial nucleic acid such as PNA or the like. The length of the polynucleotide is, for example, about 6 to 150 mer, preferably about 9 to 90 mer, and more preferably about 15 to 45 mer, although it is not particularly limited. Further, the sequence thereof is not particularly limited.

As described above, the modifying substance may contain a carbon nanotube, for example. The modifying substance may be composed of the carbon nanotube only or further may contain other substance. Examples of the other substance include a spacer, and the like. A method of binding the carbon nanotube to the binding substance is not particularly limited and conventionally known methods can be adopted. The method can be decided suitably according to types of the binding substance.

Generally, the carbon nanotube is a cylinder having a bottom surface, and may be a single layer cylinder or a multilayer cylinder in which cylinders are overlapped. In the present invention, a multilayer cylinder is preferable. The size of the carbon nanotube is not particularly limited, and the length of the single layer is, for example, about 2 to 50 nm, preferably about 3 to 30 nm, and more preferably about 5 to 15 nm, the outer diameter is, for example, about 2 to 10 nm, preferably about 3 to 9 nm, and more preferably about 5 to 8 nm, and the inner diameter is, for example, about 1 to 9 nm, preferably about 2 to 8 nm, and more preferably about 4 to 7 nm. Further, in a case of the multilayer, the overall length is, for example, about 2 to 50 nm, preferably about 3 to 30 nm, and more preferably about 5 to 15 nm.

Types of a target substance to be detected are not particularly limited and examples thereof include protein, an amine compound such as peptide, and the like; carbohydrate; polysaccharide; nucleic acid; oligonucleotide; haptene; a compound such as dioxine, endocrine-disrupting chemicals, and the like; a drug such as pesticide residue, rat poison, and the like; and the like. The binding substance is preferably a substance that specifically can bind to the target substance, although the binding substance is applicable as long as it can binds to the target substance. In a case where the target substance is a substance that can be an antigen, an example of the binding substance includes an antibody that recognizes the target substance. Further, in a case where the target substance is an antibody, an example of the binding substance includes an antigen that is recognized by the antibody.

Types of a sample to which the present invention is applied are not limited at all, and examples thereof include a sample in which the presence of a target substance is expected and a sample in which the presence of a target substance is known. Specific examples of the sample include a blood sample such as whole blood, blood plasma, blood serum, blood cell, and the like; a biological sample such as urine, spinal fluid, stool, saliva, lymph, semen, vaginal secretion such as cervical mucus, and the like; beverage; food; various drainage; soil; rainwater; river water; and the like. The sample is preferably in a form of liquid. In a case of solid, it is preferable that the sample is dissolved, dispersed, or suspended into an appropriate solvent such as water, buffer solution, or the like, or the sample is extracted with the aforementioned solvent, and a liquid fraction is subjected to the present invention as a sample.

In the present invention, a target substance and a sample to which the present invention is applied are not limited at all. However, it is preferable that the present invention is applied to detection of C-reactive protein (CRP) in blood, HbA1c, TSH, FT3, FT4, hCG, HBs antigen, HBc antibody, HCV antibody, TY antigen, antistreptolysin O (ASO), type IV collagen, matrix metalloproteinase (MMP-3), PIVAK-II, α1 microglobulin, β1 microglobulin, amyloid A (SAA), elastase 1, basic fetoprotein (BFP), candida antigen, granulocyte elastase in cervical mucus, digoxin, cystatin C, factor XIII, urinary transferring, syphilis, hyaluronic acid, fibrin monomer complex (SFMC), von Willbrand factor (factor VIII antigen), protein S, rheumatoid factor (RF), IgD, α1 acid glycoprotein (α1AG), α1 antitrypsin (α1AT), α2 macroglobulin, albumin (Alb), ceruloplasmin (Cp), haptoglobin (Hp), prealbumin, retinol-binding protein (RBP), β1C/β1A globulin (C3), μ1E globulin (C4), IgA, IgG, IgM, β lipoprotein (β-LP), apoprotein A-I, apoprotein A-II, apoprotein B, apoprotein C-II, apoprotein C-III, apoprotein E, transferring (Tf), urinary albumin, plasminogen (PLG), lipoprotein (a) (LP (a)), and the like.

Among them, CRP is protein in blood that serves as an indicator of inflammation, and the critical value for judging the presence or absence of inflammation is about 1 mg/100 mL. Therefore, particularly, accurate measurement of a concentration range in the vicinity of this critical value (for example about 0.1 to 1 mg/100 mL) is required. However, for example, it is difficult to detect such low concentration range with the conventional TIA method. Further, with respect to the conventional LA method, although a low concentration range can be detected, there is a problem of increase in background due to adsorption or the like of a latex particle. Further, generally, it is said that the ultimate upper limit concentration of CRP in blood is about 50 to 60 mg/100 mL, and it is judged as critical inflammation in a concentration range of about 4 to 20 mg/100 mL. Therefore, an accurate measurement also is required with respect to this concentration range. However, when a detection tool having the cell length of about 100 μm such as a microchip and a micro TAS is used, with respect to the conventional TIA method, there is a problem that sufficient measurement sensitivity cannot be obtained in a low concentration range of CRP of about 1 mg/100 mL. Further, with respect to the conventional LA method, there is a problem of high background, although a low concentration range of about 1 mg/100 mL can be detected. Further, with respect to the LA method, as described above, there is a problem of occurring prozone in a high concentration range. Therefore, with respect to a sample of high concentration, for example, there is a problem of requiring preparation of plural types of immobilized antibody, dilution, optimization of dilution ratio, remeasurement, and the like. As a method of avoiding the occurrence of prozone, there is a method of increasing an antigen-antibody ratio. However, in a case where a sample is undiluted, for example, there is a problem that avoidance of the occurrence of prozone is difficult with this method. In contrast, according to the present invention, for example, not only detection in a low concentration range of CRP of about 1 mg/100 mL, detection in a high concentration range of CRP of about 4 mg/100 mL to 20 mg/100 mL can be performed while avoiding prozone. Further, according to the present invention, for example, even with respect to an undiluted sample, prozone can be avoided and detection can be performed with excellent sensitivity and accuracy with respect to a wide concentration range. Moreover, according to the present invention, for example, even in a case of applying to a microchip and a micro TAS having the cell length of micrometer order, excellent sensitivity and accuracy can be maintained.

The form of a detection reagent of the present invention is not particularly limited, and may be a liquid reagent or a dried reagent. In a case of the former liquid reagent, examples of a solvent include water such as distilled water, and the like; a Good's buffer solution such as a normal saline solution, a 3-Morpholinopropanesulfonic acid (MOPS) buffer solution, and the like; a phosphate buffer solution; a tris buffer solution; and the like. The pH of the liquid reagent is, for example, about 6 to 9, although it is not particularly limited. In a case of the latter dried reagent, for example, at the time of using, the reagent may be dissolved, suspended, or dispersed into the sample by supplying the aforementioned sample or a liquid sample. Alternatively, the reagent may be dissolved, suspended, or dispersed into a solvent by supplying the solvent. As the solvent to be supplied, the solvent similar to that for the liquid regent can be used. Further, when the detection reagent is dissolved, suspended, or dispersed into the liquid sample or the solvent, the pH of the mixture thereof is preferably set at about 6 to 9. The dried reagent may be prepared by drying the aforementioned liquid reagent, for example.

As described above, the detection reagent of the present invention is applicable as long as it contains the modified binding substance. However, the detection reagent further may contain another component. The other component is not particularly limited insofar as it does not considerably adversely affect an effect of the present invention when the detection reagent of the present invention is applied to a detection method of the present invention, for example. Specific examples of the component include the aforementioned solvent; a buffer agent such as 3-Morpholinopropanesulfonic acid (MOPS), 2-Morpholinoethanesulfonic acid (MES), tris, phosphoric acid salt, and the like; a preservative agent such as sodium azide; a surfactant such as saponin, Triton (trade mark) X-100, Triton (trade mark) X-305, Triton (trade mark) X-405, Tween (trade mark) 20, Tween (trade mark) 40, Tween (trade mark) 60, Tween (trade mark) 80, and the like.

In the present invention, a production method of the modified binding substance is not particularly limited. Hereinafter, a method of producing a biotinylated binding substance and an avidin-biotinylated binding substance is described as an example using an amine compound such as an antibody as a binding substance. However, the present invention is not limited thereto.

First, biotins are brought into contact with the binding substance against a target substance. Thereby, biotins bind to the binding substance and a biotinylated binding substance is obtained. Biotination of the binding substance can be confirmed by a HABA method, for example.

At the time of biotination of an amine compound such as an antibody, for example, it is preferable to use a biotinylated reagent in which an amine reactive group is esterified to a carboxyl group of biotins. Use of such biotinylated reagent makes it possible to bind biotins stably to an amine compound by an amide binding. Generally, the binding reaction of the binding substance and the biotinylated reagent are performed by mixing them in a liquid, and leaving a thus-obtained reaction solution. Examples of the biotinylated reagent include succinimide biotin (trade name: EZ-Link (trade mark) NHS-Biotin, manufactured by Pierce Biotechnology, Inc.), and the like.

Further, as described above, when biotin is bound to the binding substance via a spacer, for example, a biotinylated reagent in which a spacer is bound to biotins may be brought into contact with the binding substance. In the biotinylated reagent, as described above, it is preferable that the spacer is bound to a carbonyl group of biotins, and the binding thereof is an amide binding. Further, in the biotinylated reagent, it is preferable that an amine reactive group is bound to the other end of the spacer, and the binding thereof is an amide binding. Use of such biotinylated reagent makes it possible to stably bind biotins to an amine compound due to an amide binding via the spacer. Examples of a biotinylated reagent to which a spacer is bound include succinimidyl-6-(biotinamido)hexanoate (trade name: EZ-Link (trade mark) NHS-LC-Biotin, manufactured by Pierce Biotechnology, Inc.), sulfosuccinimidyl-6-(biotinamido)hexanoate (trade name: EZ-Link (trade mark) Sulfo-NHS-LC-Biotin, manufactured by Pierce Biotechnology, Inc.), succinimidyl-6-[biotinamido]-6-hexanamido hexanoate (trade name: EZ-Link (trade mark) NHS-LC-LC-Biotin, manufactured by Pierce Biotechnology, Inc.), sulfosuccinimidyl-6-[biotinamido]-6-hexanamido hexanoate (trade name: EZ-Link (trade mark) Sulfo-NHS-LC-LC-Biotin, manufactured by Pierce Biotechnology, Inc.), N-[1-(biotinylamido)4,7,10-trioxatridec-13-yl]succinamic acid 2,3,5,6-tetrafluorophenyl ester (trade name: EZ-Link (trade mark) FP-PEG₃-Biotin, manufactured by Pierce Biotechnology, Inc.), sulfosuccinimidyl-2-[biotinamido]ethyl-1,3-dithiopropionate (trade name: EZ-Link (trade mark) Sulfo-NHS-SS-Biotin, manufactured by Pierce Biotechnology, Inc.), 12-biotinylamido-4,7,10-trioxadodecane acid N-hydroxysuccinimidyl ester (trade name: EZ-Link (trade mark) NHS-PEO4-Biotin, manufactured by Pierce Biotechnology, Inc.), and the like. Besides them, examples of a biotinylated reagent to which a spacer is bound include (+)-biotinyl-maleimidyl-3,6-dioxaoctanediamine (trade name: EZ-Link (trade mark) Maleimide-PEG₂-Biotin, manufactured by Pierce Biotechnology, Inc.) and (+)-biotinyl-iodoacetoamidyl-3,6-dioxaoctanediamine (trade name: EZ-Link (trade mark) Iodoacetyl-PEG2-Biotin, manufactured by Pierce Biotechnology, Inc.), which bind biotin to a molecule having a thiol group; (+)-biotinyl-3,6-dioxaoctanediamine (trade name: EZ-Link (trade mark) Amine-PEG2-Biotin, manufactured by Pierce Biotechnology, Inc.), (+)-biotinyl-3,6,9-trioxaundecanediamine (trade name: EZ-Link (trade mark) Amine-PEG3-Biotin, manufactured by Pierce Biotechnology, Inc.), and 5-biotinamido pentylamine (trade name: EZ-Link (trade mark) Pentylamine-Biotin, manufactured by Pierce Biotechnology, Inc.), which bind biotin to a molecule having a carboxyl group; biotinylated psoralen (trade name: EZ-Link (trade mark) Psoralen-PEG3-Biotin, manufactured by Pierce Biotechnology, Inc.) which labels a nucleic acid such as DNA, RNA, etc. and protein with biotin; dimer biotin (trade name: EZ-Link (trade mark) PEG5-Biotin Dimer, manufactured by Pierce Biotechnology, Inc.), which further labels a biotin binding substance with biotin; and the like. For example, these biotinylated reagents include the configuration of the aforementioned spacers (1) to (12), respectively.

In the reaction solution, the ratio between the binding substance and the biotins (or the biotinylated reagent) is not particularly limited. Preferably, about 0.1 mmol/L to 4 mmol/L of biotins are added to 1 mmol/L of binding substance, and more preferably, about 0.5 mmol/L to 2 mmol/L of biotins are added to 1 mmol/L of binding substance. Specifically, in a case where the binding substance is an antibody, preferably, about 0.1×10⁻⁴ mmol/L to 0.1 mmol/L of bitions are added to 1 mg of antibody, and more preferably, about 0.2×10⁻⁴ mmol/L to 0.05 mmol/L of bitions are added to 1 mg of antibody. For example, the concentration of biotins in the reaction solution is preferably about 0.1×10⁻³ mmol/L to 0.01 mmol/L, although it is not particularly limited. A solvent for the reaction solution is not particularly limited and examples thereof include water such as distilled water and the like; a buffer solution such as a phosphate buffer solution and the like; and the like. The pH of the solvent is, for example, about 6 to 9. For example, treatment is carried out at a room temperature (for example, about 10° C. to 40° C.) for about 5 to 120 minutes, although a treatment condition is not particularly limited.

Next, unreacted biotins are removed from the reaction solution and a fraction containing a biotinylated binding substance is collected. A method of removing unreacted biotins is not particularly limited and may be performed by centrifugal separation using a desalting column. Examples of the column include a product under the name of Zeba (trade mark) Desalt Sipn columns manufactured by Pierce Biotechnology, Inc, and the like.

With respect to the fraction containing a biotinylated binding substance, for example, concentration treatment may be performed. The concentration may be performed by centrifugal separation using an ultrafiltration filter, for example. In a case where the binding substance is protein such as an antibody and the like, a molecular weight cut off of the ultrafiltration filter is, for example, about 10 K to 500 K.

With respect to a biotinylated binding substance contained in the concentrated solution, it is preferable that a biotinylated ratio is confirmed. The biotinylated ratio can be confirmed by a HABA method using a commercially available kit, for example.

Further, when an avidin-biotinylated binding substance is prepared, preferably, in advance of the reaction with avidin that is a next process, an amount of the binding substance in the concentrated solution is measured. The measurement method is not particularly limited and can be decided suitably according to types of the binding substance. In a case where the binding substance is protein such as an antibody and the like, an example of the measurement method includes an optical measurement method such as an absorbance measurement at the wavelength of 280.

Subsequently, when an avidin-biotinylated binding substance is prepared, avidins are brought into contact with a biotinylated binding substance that is previously obtained. Thereby, avidins further are bound to biotins in a biotinylated binding substance, and thus the avidin-biotinylated binding substance is obtained. Binding of avidins to a biotinylated binding substance can be performed by mixing the biotinylated binding substance and avidins in a liquid, and leaving a thus obtained reaction solution.

In the reaction solution, the ratio between the biotinylated binding substance and the avidins is not particularly limited. Preferably, about 0.1 mmol/L to 4 mmol/L of avidins are added to 1 mmol/L of biotinylated binding substance, and more preferably, about 0.5 mmol/L to 2 mmol/L of avidins are added to 1 mmol/L of biotinylated binding substance. Specifically, in a case where the binding substance is an antibody, it is preferable that about 1×10⁻⁶ mmol/L to 100 mmol/L of avidins are added to 1 mg of biotinylated antibody. For example, the concentration of avidins in the reaction solution is preferably about 1×10⁻⁴ mmol/L to 10 mmol/L, and more preferably about 1×10⁻² mmol/L to 5 mmol/L, although it is not particularly limited. A solvent for the reaction solution is not particularly limited and examples thereof include solvents similar to those used in the biotinylated treatment. For example, treatment is carried out at a room temperature (for example, about 10° C. to 40° C.) for about 5 to 120 minutes, although a treatment condition is not particularly limited.

Then, unreacted avidins are removed from the reaction solution and a fraction containing an avidin-biotinylated binding substance is collected. A removing method of the unreacted avidins is similar to that of the unreacted biotins, for example.

According to the method described above, for example, an avidin-biotinylated binding substance, in which the molar ratio between biotins and avidins is about 1:1, can be produced efficiently.

However, a production method of an avidin-biotinylated binding substance is not limited to the aforementioned method. For example, the avidin-biotinylated binding substance may be produced by preliminarily reacting avidins with biotins, forming avidin-biotin complex, and reacting this complex with the binding substance.

Further, in a case where the modifying substance is the carbon nanotube, for example, as described in JP2002-503204 A, a carbon nanotube binding substance can be produced by binding a functional group on the surface of the carbon nanotube and, therethrough, binding with the binding substance such as an antibody or the like. Moreover, in a case where the modifying substance is a nucleic acid, for example, as described in WO2004/111232, a nucleic-acid bound substance can be produced by binding the 5′ end of polynucleotide to a site of an antibody other than an antigen recognition site.

### <Detection Method>

As described above, the detection method of the present invention is a method of detecting a target substance in a sample using a binding substance that binds to the target substance, comprising the following (A) and (B):
(A) bringing the binding substance into contact with the sample, and forming an aggregate by binding of the binding substance and the target substance in the sample; and
(B) detecting the aggregate, wherein
   the binding substance is a binding substance (modified substance) in which modifying substances having the maximum diameter of about 50 nm or less are bound.

According to the present invention, as described above, a target substance can be detected with excellent accuracy and sensitivity by using a modified binding substance in which the aforementioned modifying substances are bound. Specifically, the method of the present invention allows measurement in a low concentration range that hardly was measured with the TIA method. Further, background can be reduced as compared to the LA method and the occurrence of prozone in a high concentration range can be avoided effectively. Particularly, according to the present invention, since occurrence of prozone can efficiently be avoided without diluting a sample, in contrast to the conventional method, detection can be performed using an undiluted sample. Therefore, according to the present invention, a target substance can be detected with better accuracy and sensitivity as compared to the conventional LA method and the TIA method. Further, according to the present invention, even in a case where a formed aggregate is detected by an optical method with the cell length of micrometer order, a target substance can be detected with respect to a concentration range, which could not be measured with the conventional method, for example. As described above, according to the present invention, a target substance can be detected with excellent accuracy and sensitivity with respect to a concentration range, which could not be measured with the conventional method, for example. As can be seen from the above, the present invention is a very useful new method in an analysis and clinical field.

In the present invention, the modified binding substance is as described above. Further, in the detection method of the present invention, the detection reagent of the present invention can be used as the modified binding substance.

The detection method of the present invention may be either wet type or dry type. In the case of the wet type, in the (A), for example, a liquid detection reagent of the present invention is used as the modified binding substance, and the liquid reagent and a liquid sample are mixed. Further, in the case of the dry type, for example, a dried detection reagent of the present invention is used as the modified binding substance, a liquid sample is added to the dried reagent, and the modified binding substance is dissolved, suspended, or dispersed into the liquid sample. In the latter case, the dried reagent may be placed in a predetermined region of a tool such as a cell, a chip, a microchip, a micro tube, a micro reactor, a micro TAS, a test tube, a test piece, and the like. In this case, for example, the liquid sample may be supplied to a region of the tool where the dried reagent is placed. A method of supplying the liquid sample is not limited at all. For example, the liquid sample may be added directly to the region where the dried reagent is placed or may be supplied to the region through a channel or the like. A detection tool in which the detection reagent of the present invention is placed will be described later in details.

In the (A), an amount of the modified binding substance to be brought into contact with the sample is not particularly limited, and, for example, is decided suitably according to types of the target substance, types of the binding substance, connectivity of the target substance and the binding substance, types of the sample, and the like. For example, when the upper limit amount (upper limit concentration) of the target substance presented in a sample is well known or when the upper limit of a detection amount (concentration) of the target substance is set, it is assumed that the target substance of the upper limit amount (upper limit concentration) is presented, and the modified binding substance of enough amounts for detecting the target substance of the upper limit amount may be used. With respect to this point, specifically, measurement of CRP in blood using an anti-CRP antibody (biotinylated anti-CRP antibody), to which biotin is bound, as the modified binding substance is explained as an example. As described above, CRP is protein in blood serving as an indicator of an inflammation, and when a concentration thereof is in the upper limit of about 4 mg to 20 mg/100 mL, it is considered as critical inflammation. Therefore, for example, in a case where undiluted blood (or undiluted blood plasma) is used as a sample, it is preferable to contact a sample with a biotinylated anti-CRP antibody of enough amounts for detecting CRP of the upper limit concentration of about 4 mg to 20 mg/100 mL.

As described above, the amount of the modified binding substance is not particularly limited. Generally, the higher the ratio of a binding substance such as an antibody or the like relative to a target substance such as an antigen or the like is, the higher the measurement accuracy. Therefore, for example, it is preferable that the ratio of the modified binding substance amount (for example, modified antibody amount) is high relative to an expected amount of a target substance in a sample.

A treatment condition of the (A) is not particularly limited. A treatment temperature is, for example, about 5° C. to 60° C., and preferably about 20° C. to 35° C. A treatment time is, for example, about 1 to 30 minutes, and preferably about 2 to 20 minutes.

An aggregate formed by binding of the target substance and the modified binding substance in the (A) is detected in the following (B).

A detection method of the aggregate is not particularly limited, and conventionally known methods can be used. An example of the method includes an optical detection method, and specific examples thereof include measurement of transmitted light intensity, measurement of scattered light intensity, and the like. As a method of detecting a target substance by an optical detection of an aggregate, generally, a turbidimetry performing measurement of transmitted light and a nephelometry performing measurement of scattered light are known. Since the turbidimetry and the nephelometry each have a different concept, use of a reagent for turbidimetry to nephelometry or use of a reagent for nephelometry to turbidimetry is considered to be difficult. In fact, even in a case where the same target substance is detected, a reagent has a different component and composition in accordance with each method. In contrast, according to the detection reagent of the present invention, an aggregate formed in the (A) can be detected by either transmitted light or scattered light in the (B). Therefore, the detection reagent, the detection tool, and the detection method of the present invention are applicable to both of the turbidimetry using the existing transmitted light measurement device and the nephelometry using the existing scattered light measurement device.

Since the intensity of transmitted light described as specific examples and absorbance calculated from intensity of scattered light or intensity of transmitted light show correlation with an aggregate amount, by measuring them, an aggregate concentration in a reaction system can be calculated, for example. Further, since a concentration of this aggregate is correlated with a concentration of a target substance in a sample, as a result, a concentration and an amount of a target substance in a sample can be calculated. The reaction system means, for example, a sample that is in contact with the modified binding substance or a mixture containing it (for example, a mixed liquid). Further, it may be a wet type reaction system (reaction solution) or a dry type reaction system (hereinafter, the same applies). The wavelength of light to be irradiated is, for example, about 260 nm to 1100 nm, and preferably about 310 nm to 400 nm. This measurement can be performed with a commercially available spectrophotometer, a scattered light measurement device, and the like. The cell length at the time of measurement is not particularly limited. For example, measurement with excellent sensitivity can be performed not only with the general cell length such as about 10 mm, but also with the cell length (for example, about 10 μm to 1000 μm, preferably about 100 μm) of micrometer order of a microchip, a micro TAS, and the like which will be described later. In the present invention, “the cell length” refers to the length of a reaction system in a direction of light path of irradiating light in a reaction system in which light is irradiated at the time of measurement.

Above all, in the detection method of the present invention, it is preferable to measure scattered light. In particular, it is preferable to measure scattered light by the following method or using a scattered light measurement device, or the like. For example, since scattered light can be sensed efficiently by such method or using such device, measurement accuracy by a detection method of the present invention further can be increased.

An example of a scattered light measurement method is as follows. The scattered light measurement method of this embodiment includes a light irradiating process for irradiating light to a reaction system from a light source and a light receiving process for receiving the irradiating light irradiated to the reaction system at a light receiving portion, for example. In the light receiving process, direct light transmitted through the reaction system out of the irradiating light is blocked, and the scattered light is received at the light receiving portion. Generally, the scattered light means light that changes its path in a medium. However, in the present invention, the scattered light includes an extended meaning such as reflected light, refracting light, and the like.

In this scattered light measurement method, efficient scattered light receiving is realized without difficulty by blocking direct light that is transmitted through the reaction system. The way of blocking the direct light is not limited at all. Examples of the method of blocking the direct light include a method of reflecting or absorbing the direct light transmitted through a sample before the direct light reaches a light receiving portion.

This scattered light measurement method is realized by using the scattered light measurement device that is described below and a detection tool that will be described later, for example. Hereinafter, an embodiment of the scattered light measurement device is described. However, the present invention is not limited thereto.

### <Scattered Light Measurement Device>

A scattered light measurement device of this embodiment includes a light source irradiating light, a light receiving portion receiving the light, a sample placement portion to which a sample is placed, and a light blocking portion blocking the light. The light receiving portion is disposed in a direction of light path of the irradiating light from the light source, the sample placement portion is disposed between the light source and the light receiving portion, and the light blocking portion is disposed in the intermediate position of the light path of direct light transmitted through the sample between the sample placement portion and the light receiving portion. In the present invention, a sample to be placed at the sample placement portion may be a sample before being in contact with a detection reagent of the present invention or a sample (reaction system) after being in contact with the detection reagent, for example. The present invention is applicable as long as a target substance in the sample and the modified binding substance in the detection reagent are reacted at the time of measurement of the scattered light. Further, a sample to be irradiated with the light from the light source is, for example, a sample that is in contact with a detection reagent of the present invention or a mixture containing the same, and the sample is the aforementioned reaction system.

According to the scattered light measurement device of this embodiment, for example, unlike the conventional scattered light measurement device, it is not required to move a light receiving portion to a wanted angle or to set plural light receiving portions at plural angles for receiving scattered light. Further, according to the scattered light measurement device of the present invention, for example, positions of a light source, a light receiving portion, and a sample placement portion can be fixed. Since a positional relationship can be stabilized in this manner, for example, the reliability of measurement accuracy can be maintained.

Preferably, the light blocking portion absorbs or reflects light, for example. Specifically, the light blocking portion preferably contains a light absorptive material or a light reflective material, and particularly preferably contains a light reflective material. Alternatively, the light blocking portion may contain both the light absorptive material and the light reflective material. The light absorptive material is not particularly limited and examples thereof include a black paint; a black powder (inorganic powder) such as copper(II) oxide, cobalt oxide, and the like; a carbon; a black plastic; black alumite; and the like. The light reflective material is not particularly limited and examples thereof include a mirror material and a mirror finishing material; a white paint; a white powder such as titanium oxide, aluminum oxide, and the like; and the like. Examples of the mirror material and the mirror finishing material include ceramic, metal, and the like. The whole light blocking portion may contain the aforementioned light absorptive material or the aforementioned light reflective material. Alternatively, the light blocking portion may be formed by coating the aforementioned material on the surface of a substrate or applying a film containing the aforementioned material to the surface of a substrate. Examples of the quality of material of a substrate into which the aforementioned materials are incorporated in the former case and the quality of material of a substrate coated with the aforementioned materials in the latter case include permeable polymer such as acrylic, and the like. Further, with respect to a cover member covering the light receiving portion, a region required to be blocked from the direct light may contain the aforementioned materials. In this case, it is preferable that the cover member except for the light blocking portion may be composed of the materials through which the light is transmitted, which will be described later. Alternatively, for example, with respect to the surface of the cover member covering the light receiving portion, the region required to be blocked with the direct light may be coated with the aforementioned material or applied with a film containing the aforementioned material. It is preferable that the cover member is formed of materials through which the light is transmitted.

Preferably, in particular, the light blocking portion reflects light. When the light is reflected at the light blocking portion, since the sample is irradiated with reflected light, the sample is irradiated with the light more than once. Thereby, scattered light received at a light receiving portion further can be increased.

The size and the shape of the light blocking portion are not particularly limited as long as the direct light transmitted through the sample can be blocked. For example, the light blocking portion may have the size and the shape that allow blocking of scattered light at an unwanted angle in addition to blocking the direct light.

Further, in addition to the light blocking portion for blocking the direct light, the scattered light measurement device of this embodiment further may comprise a light blocking portion for blocking scattered light at an unwanted angle that is transmitted through the sample. Thereby, for example, only scattered light of the wanted angle selectively can be received.

With respect to the scattered light measurement device of this embodiment, for example, it is preferable that a sample holding tool having a sample holder is disposed at the sample placement portion at the time of measurement. In this case, for example, in advance of the aforementioned light irradiating process, after supplying (placing) the sample to the sample holder of the sample holding tool, the sample holding tool is disposed at the sample placement portion of the scattered light measurement device. Then, the sample in the sample holding tool is irradiated with the light (emitted light) from the light source. Alternatively, in advance of the light irradiating process, after disposing the sample holding tool to the sample placement portion of the scattered light measurement device, the sample may be supplied (placed) to the sample holder of the sample holding tool. A sample to be supplied to the sample holding tool may be a sample before being in contact with a detection reagent of the present invention or a sample (reaction system) after being in contact with the detection reagent, for example. In the former case, for example, a sample holding tool (for example, a detection tool of the present invention that will be described later) to which the detection reagent preliminarily is placed is provided, and a sample and a detection reagent may be in contact with each other in the sample holding tool by supplying the sample to the sample holding tool.

The sample holding tool may have a configuration of providing a sample holder having a concave cross-sectional shape, for example. Alternatively, the sample holding tool may have a configuration of providing a channel, for example. As a specific example of the configuration, the sample holding tool may be provided with a sample introduction port, a channel, and a sample holder, the introduction port and the sample holder being in communication with each other via the channel. Alternatively, the sample holding tool may include an introduction port and a channel, the introduction port and the channel may be in communication with each other, and a part of the channel may also serve as a sample holder. In a case where the sample holding tool is provided with the detection reagent, for example, a site to which the detection reagent is placed may be the sample holder or the channel between the introduction port and the sample holder, although it is not particularly limited. The sample holding tool is not particularly limited, and conventionally known tools such as a cell, a chip, a microchip, a micro tube, a bioreactor, and the like can be used.

In the sample holding tool, the length of the sample holder in the direction of light path of the irradiating light to the sample is, for example, in the range of about 10 μm to 1000 μm, preferably in the range of about 50 μm to 500 μm, and more preferably in the range of about 100 μm to 400 μm, although it is not particularly limited. This length may be referred to as the cell length or the light path length, for example.

Further, with respect to the scattered light measurement device, for example, a sample may directly be supplied (placed) to the sample placement portion. In this case, examples of the sample placement portion include a channel and a concave portion, which are formed in the scattered light measurement device, for holding a sample. The shape and the size of the sample placement portion are not particularly limited. When the sample is placed at the sample placement portion, the length of a region for sample in the direction of light path of the irradiating light from the light source, i.e., the thickness of the sample in the direction of light path placed in the device, is, for example, in the range of about 10 μm to 1000 μm, preferably in the range of about 50 μm to 500 μm, and more preferably in the range of about 100 μm to 400 μm.

In the scattered light measurement device, types of the light receiving portion are not particularly limited, and an example thereof includes a light receiving element such as a photodiode, a phototransistor, a photomultiplier tube, and the like. The number of the light receiving element in the light receiving portion is preferably few, and particularly preferably one, although it is not particularly limited.

In the scattered light measurement device, types of the light source are not particularly limited, and examples thereof include an LED, an LD, a laser, a mercury lamp, a halogen lamp, a xenon lamp, a tungsten lamp, and the like. The wavelength thereof is, for example, in the range of about 260 nm to 1100 nm, preferably in the range of about 310 nm to 650 nm, and more preferably in the range of about 365 nm to 415 nm, although it is not particularly limited. The diameter of light irradiated from the light source can be adjusted by an aperture of a light emitting portion, for example. For example, the light emitting portion and the light source are in communication with each other via an optical fiber, or the like.

In the scattered light measurement device, examples of a light source include a small-diameter light source (also may be referred to as “point light source”) and a line light source. In a case of the small-diameter light source, since scattered light can be received circumferentially, sensitivity can be improved, for example. Further, in a case of the line light source, sensitivity can be improved by having a slit structure, for example.

The scattered light measurement device further may include an amplifier for amplifying voltage generated due to receiving of scattered light at the light receiving portion, an I/V circuit which changes current change to voltage change, an AD converter which converts voltage to digital value, or the like. Further, the scattered light measurement device further may include, for example, a CPU for converting current value to light intensity (scattered light intensity), adjusting emission of the light source, and converting current digital value to light intensity (scattered light intensity).

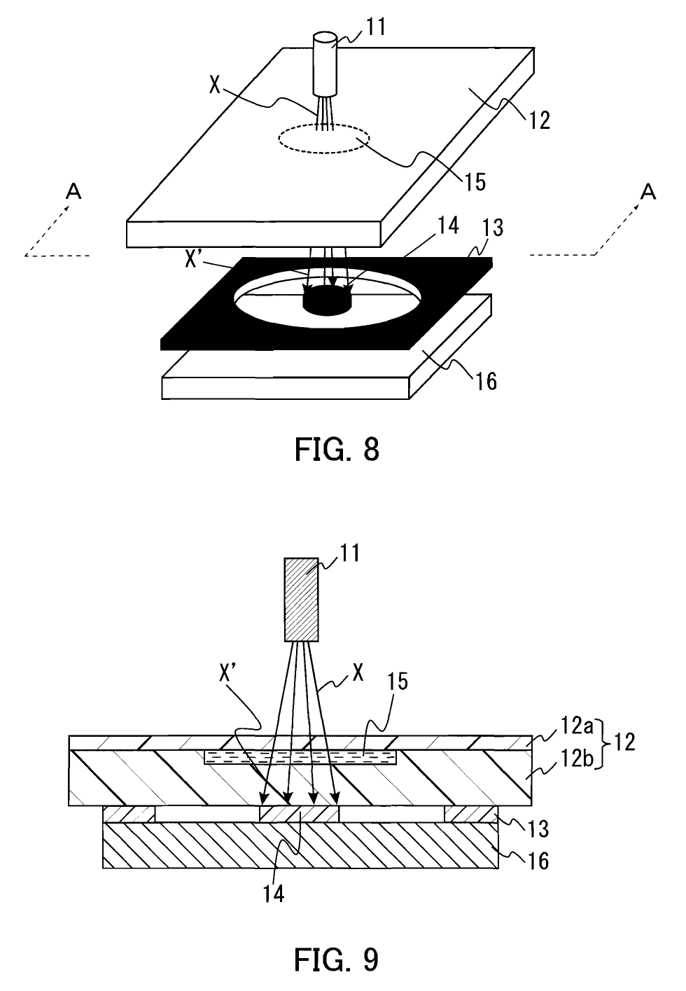

Hereinafter, with respect to the scattered light measurement device, further specific examples are explained using FIGS. 8 to 10. These embodiments are examples of a scattered light measurement device using a sample holding tool diposed at a sample placement portion. However, in the present invention, a scattered light measurement device is not limited to these embodiments.

### Embodiment 1

An embodiment 1 is an example of a scattered light measurement device provided with a small-diameter light source as a light source.

FIG. 8 is a perspective view of a scattered light measurement device to which a sample holding tool is provided. FIG. 9 is a cross-sectional view of FIG. 8 in the direction of A-A. FIG. 8 is a schematic view in which each component is shown separately for convenience sake. As shown in FIGS. 8 and 9, the scattered light measurement device is provided with a light source 11, a light receiving portion 16, a first light blocking portion 14, and a second light blocking portion 13. In this Embodiment, the first light blocking portion 14 is a member for blocking direct light transmitted through a sample and the second light blocking portion 13 is a member for blocking scattered light of unwanted angle transmitted through the sample. The light receiving portion 16 is disposed opposite to the light source 11. Specifically, the light receiving portion 16 is disposed in a direction of light path of irradiating light X from the light source 11. Further, the first light blocking portion 14 and the second light blocking portion 13 are disposed on the surface of the light receiving portion 16 at the light source 11 side. In the scattered light measurement device, at the time of using, a sample holding tool 12 that holds a sample at a sample holder 15 is disposed on the light receiving portion 16 via the first light blocking portion 14 and the second light blocking portion 13. In the scattered light measurement device, a site where the sample holding tool 12 is disposed is a so-called sample placement portion. The sample holding tool 12 includes a support substrate 12b and a cover substrate 12a. On the surface of the support substrate 12b, a concave groove is formed as the sample holder 15, and the surface of the support substrate 12b on which the groove is formed is covered with the cover substrate 12a. In the scattered light measurement device, the first light blocking portion 14 is disposed in the intermediate position of the light path of direct light X′, transmitted through the sample 15, out of irradiating light X from the light source 11. Further, the second light blocking portion 13 is disposed in the intermediate position of the light path of scattered light, transmitted through the sample and changed in path to an unwanted angle, out of the irradiating light X from the light source 11.

In the scattered light measurement device, the shape of the first light blocking portion 14 is not particularly limited and can be decided suitably according to types of the light source. When the light source is a small-diameter light source as in the case of this Embodiment, the planar shape of the first light blocking portion 14 is preferably circular. In this state, the planar shape means a surface that is irradiated with the direct light transmitted through the sample. Further, in the scattered light measurement device, it is preferable that the center of the first light blocking portion 14 is opposed to the center of the irradiating light from the small-diameter light source.

The size of each part in the scattered light measurement device is not particularly limited and can be decided suitably according to the size of the sample holding tool 12, the size of the sample holder 15 in the sample holding tool 12, and the like. Further, in a case where the scattered light measurement device of the present invention is produced using a light intensity measurement device such as the existing spectrophotometer or the like, for example, the size of the light blocking portion and the like can be set suitably according to the size of each part of the spectrophotometer. Hereinafter, the size of each part of the scattered light measurement device is described by assuming a sample holding tool to be a general microchip. However, the following description is an example and the present invention is not limited thereto.

The size of the first light blocking portion 14 is not particularly limited and can be decided suitably according to the diameter of the irradiating light from the small-diameter light source 11, the distance between a position of emitting the irradiating light from the small-diameter light source 11 and the surface of the sample at the light source side, the thickness of the sample holding tool, and the like, for example. As a specific example, the ratio between the diameter of the irradiating light from the small-diameter light source and the diameter of the planar surface of the light blocking portion is preferably about 1:2 to 100, more preferably about 1:20 to 100, and particularly preferably about 1:40 to 60. The term of the diameter of the irradiating light means, for example, the diameter of the irradiating light at the time of emitting.

The thickness of the first light blocking portion 14 is not particularly limited as long as it can block the light. As shown in FIGS. 8 and 9, in a case where a member for blocking the light is provided as the light blocking portion, the upper limit of the thickness is, for example, preferably about 1 mm or less and particularly preferably about 0.1 mm, and the lower limit of the thickness is, for example, preferably about 0.05 mm (about 50 μm) or less. Further, in a case of applying a seal or coating, the upper limit of the thickness is preferably about 1 mm or less and particularly preferably about 0.1 mm, and the lower limit of the thickness is, although it is not particularly limited, for example, about 0.05 mm (about 50 μm).

Generally, the diameter of the irradiating light (for example, the irradiating light at the time of emitting) from the small-diameter light source is preferably about 20 to 800 μm, more preferably about 20 to 400 μm, and particularly preferably about 40 to 100 μm, although it is not particularly limited.

Generally, the distance between a position of emitting the irradiating light from the small-diameter light source 11 and the surface of the sample 15 at the light source side or the surface of the sample holding tool 12 at the light source side is preferably about 0 to 10 mm, more preferably about 1 to 5 mm, and particularly preferably about 1 to 3 mm, although it is not particularly limited.

Generally, the distance between the surface of the sample 15 at the light source side and the surface of the light receiving portion 16 is preferably about 1 to 50 mm, more preferably about 1 to 20 mm, and particularly preferably about 2 to 5 mm, although it is not particularly limited.

The size, the shape, the material, and the like of the sample holding tool 12 are not particularly limited, and a common microchip can be used. In a case of the common microchip, with respect to the size thereof, for example, the overall length is about 20 to 200 mm, the overall width is about 20 to 150 mm, and the overall thickness is about 0.1 to 10 mm. The thickness of the sample holder 15, i.e., the length (cell length) of the sample holder 15 in the direction of light path of the irradiating light from the light source 11 is, for example, in the range of about 10 to 1000 μm and preferably about 100 μm.

The shape of the sample holder 15 in the sample holding tool 12 is not particularly limited. For example, in a case where the small-diameter light source is used, the planar shape thereof is preferably circular. The planar surface means a surface that is irradiated with the irradiating light X from the light source 11 in the sample holder 15. For example, the diameter of the planar surface of the sample holder 15 is preferably about 0.1 to 10 mm, more preferably about 0.5 to 1.2 mm, and particularly preferably about 0.7 to 0.8 mm. Further, for example, the volume of the sample held by the sample holder 15 is preferably about 1 to 1000 μL.

The material of the sample holding tool 12 is not particularly limited. Generally, in order to reduce background, the material is preferably a light transmissive material. Examples of the material include a polymer such as polymethylmethacrylate (PMMA), polydimethylsiloxane (PDMS), polyethylene terephthalate (PET), polystyrene (PS), and the like; a glass; and the like. The specific shape of the sample holding tool is similar to a detection tool of the present invention that will be described later.

Using this scattered light measurement device, for example, the aforementioned scattered light measurement method is performed as follows. First, as described above, the sample holding tool 12 is disposed at a predetermined position after supplying the sample 15 thereto. The sample 15 may be a sample (reaction system) after being in contact with a detection reagent or a sample before being in contact with a detection reagent. In the latter case, as described above, preferably, the detection reagent is placed at the sample holding tool 12 and the sample and the detection reagent come into contact with each other by supplying the sample, for example. Further, the irradiating light X is emitted from the light source 11 and the sample 15 at the sample holding tool 12 is irradiated with the light. Then, the direct light X′ transmitted through the sample 15 out of the irradiating light X irradiated to the sample 15 is blocked at the first light blocking portion 14 and prevented from reaching the light receiving portion 16 located below. Further, the scattered light, transmitted through the sample 15 and changed in path to an unwanted angle, out of the irradiating light X irradiated to the sample 15, is blocked at the second light blocking portion 13 and prevented from reaching the light receiving portion 16 located below (not shown). In contrast, the scattered light, transmitted through the sample 15 and reaching a region between the first light blocking portion 14 and the second light blocking portion 13, out of the scattered light occurring due to contact with the sample 15, is received at the light receiving portion 16 located below. In this manner, the wanted scattered light can be received efficiently by blocking the direct light and further blocking the unwanted scattered light. The second light blocking portion 13 is optional and does not limit the present invention.

Out of the light blocking portions, particularly, the first light blocking portion 14 preferably reflects the light. In this case, in FIG. 9, the direct light X′ transmitted through the sample 15 changes its path in a direction of the sample 15 due to reflection when the direct light X′ reaches the first light blocking portion 14. When this reflected light is irradiated on the sample 15, the scattered light further occurs. Therefore, an amount of the scattered light further can be increased. Further, in a case where the first light blocking portion 14 reflects the light, a light receiving portion (not shown) may further be provided at the side of irradiating the irradiating light. By further providing the light receiving portion in this manner, for example, the direct light, transmitted again through the sample 15, out of the direct light X′ transmitted through the sample 15 can be received. Thereby, at the light receiving portion 16, only desired scattered light can be received. At the other light receiving portion (not shown), the direct light (so called transmitted light) transmitted through the sample can be received.

In this Embodiment, members 13 and 14, which are composed of the light blocking portion only, are provided above the light receiving portion 16. However, the present invention is not limited thereto. For example, a cover having a light blocking portion such as described above may be provided above the light receiving portion 16. This can be produced by providing a cover member through which the light is transmitted, and by applying a film for blocking the light or coating the material for blocking the light to a region where the light should be blocked.

### Embodiment 2

Embodiment 2 is an example of a scattered light measurement device provided with a line light source as a light source.

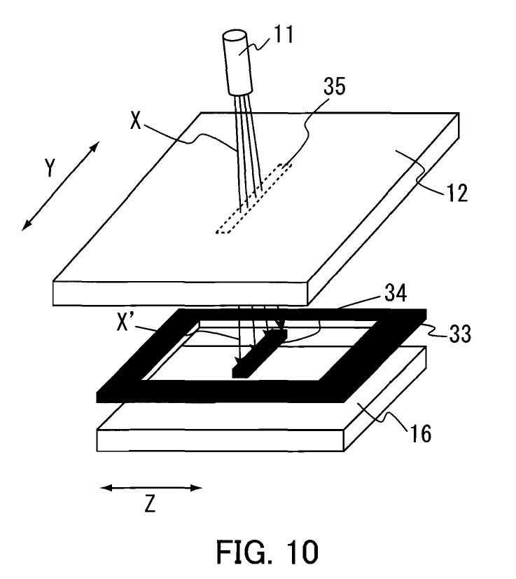

FIG. 10 is a perspective view of a scattered light measurement device in which a sample holding tool is provided, and is a schematic view in which each component is shown separately for convenience sake. Each component of Embodiment 2 is the same as that of the Embodiment 1 unless otherwise described.

As shown in FIG. 10, the scattered light measurement device of Embodiment 2 has the configuration similar to that of Embodiment 1 except that a light source 11 is a line light source, the planar shape of a first light blocking portion 34 and the shape of the seating rim of a second light blocking portion 33 are rectangular, and the planar shape of a sample holder 35 in a sample holding tool 12 is rectangular. In FIG. 10, an arrow Y shows a longitudinal direction and an arrow Z shows a perpendicular direction relative to the longitudinal direction.

In the scattered light measurement device, the shape of the first light blocking portion 34 is not particularly limited. When the light source is the line light source as in the case of this Embodiment, the planar shape of the light blocking portion is preferably rectangular. The planar surface means a surface that is irradiated with the direct light transmitted through the sample. Further, in the scattered light measurement device, it is preferable that the longitudinal direction (Y direction) of the first light blocking portion 34 is opposed to the longitudinal direction (Y direction) of the irradiating light from the line light source.

The size of the first light blocking portion 34 is not particularly limited. For example, the ratio between the length of the longitudinal direction (Y direction) of the irradiating light from the line light source and the length of the longitudinal direction (Y direction) of the light blocking portion is preferably about 1:1 to 100, more preferably about 1:1 to 10, and particularly preferably about 1:1 to 3. Further, for example, the ratio between the perpendicular direction (Z direction) relative to the longitudinal direction of the irradiating light and the length of the perpendicular direction (Z direction) of the light blocking portion is preferably about 1:1 to 100, more preferably about 1:1 to 10, and particularly preferably about 1:2 to 5. In the present invention, the length of the longitudinal direction (Y direction) of the irradiating light from the line light source means the length of the irradiating light at the time of emitting.

Generally, the length of the irradiating light (length in Y direction) from the line light source at the time of emitting is preferably about 10 to 3000 μm, more preferably about 100 to 2000 μm, and particularly preferably about 800 to 1500 μm, although it is not particularly limited.

The shape of the sample holder 35 in the sample holding tool 12 is not particularly limited. For example, in a case of the line light source, the planar shape thereof is preferably rectangular. In the sample holder 35, the planar surface means, for example, a surface that is irradiated with the irradiating light X from the light source 11. For example, in the sample holder 35, the length of the longitudinal direction (Y direction) is preferably about 10 to 150 mm, more preferably about 20 to 100 mm, and particularly preferably about 50 to 80 mm. Further, for example, in the sample holder 35, the length of the perpendicular direction (Z direction) relative to the longitudinal direction is preferably about 10 to 150 mm, more preferably about 20 to 100 mm, and particularly preferably about 50 to 80 mm.

### <Detection Tool>

The detection tool of the present invention is a detection tool used for the detection method of the present invention described above. The detection tool includes a main body and a detection reagent of the present invention. The detection reagent is placed in the main body.

A detection chip of the present invention is applicable as long as it contains the detection reagent of the present invention, and other configurations and conditions are not limited at all. The shape of the tool is not limited at all, and examples thereof include a cell, a chip, a microchip, a micro tube, a micro reactor, a micro TAS, a test tube, a test piece, and the like.

Preferably, the detection reagent of the present invention in a dried state is placed in the detection tool of the present invention. As described above, the detection reagent of the present invention can be stored stably for a long period of time in a dried state. Therefore, the detection tool of the present invention in which the dried detection reagent is placed is also excellent in stability in addition to the detection sensitivity described above. Hereinafter, such detection tool also may be referred to as a dry type detection tool of the present invention.

Since the detection tool of the present invention is excellent in stability as described above, for example, it can be stored under the condition of about 2 to 8° C. for more than about 12 months from the time of production.

In a case of the dry type detection tool, for example, at the time of using, by addition of a liquid sample, a biotinylated binding substance of the detection reagent can be dissolved or dispersed into the liquid sample. Thereby, a target substance in the liquid sample and the biotinylated binding substance of the detection reagent are bound to form an aggregate. Alternatively, with respect to the dry type detection tool, for example, at the time of using, by adding a solvent in advance of the addition of the liquid sample, the detection reagent can be dissolved or dispersed into the solvent. Further, by further adding the liquid sample, the target substance in the liquid sample and the biotinylated binding substance of the detection reagent are bound to form an aggregate.

The configuration of the detection tool of the present invention is not particularly limited as described above. The detection tool may have a configuration that the main body includes a sample inlet, a sample holder and a channel, and the sample inlet and the sample holder are in communication with each other via the channel, for example. The sample holder is a region in which a sample being in contact with a target substance is held. For example, the sample holder also may be referred to as a light irradiating portion or a detection portion because the sample held at the sample holder is irradiated with light at the time of optical detection of the aggregate. At the time of detection, in such detection tool, the detection reagent may be placed in the channel or the sample holder. With respect to such detection tool, for example, when a liquid sample is introduced from the inlet, the liquid sample reaches the sample holder through the channel. In a case where the detection reagent is placed at the sample holder, for example, a liquid sample before being in contact with the detection reagent is supplied. When the liquid sample reaches the sample holder through the channel, at the sample holder, the detection reagent is dissolved or dispersed into the liquid sample and thereby formed an aggregate. At this time, the sample holder also may be referred to as a reaction portion. On the other hand, in a case where the detection reagent is placed in the channel, for example, when the liquid sample is passed through the channel, the detection reagent is dissolved or dispersed into the liquid sample and then reaches the sample holder. In this case, for example, an aggregate is formed in the liquid sample transferred from the channel to the sample holder. For example, detection of the aggregate can be performed by irradiating the light to the sample holder of the detection tool, and measuring intensity of the transmitted light transmitted through the sample (reaction system) and intensity of the scattered light occurred due to irradiation of the light to the sample (reaction system). According to the present invention, for example, the detection reagent (biotinylated binding substance) of the present invention to be placed hardly is adsorbed by the sample holder, the channel, and the like. Therefore, as compared to the conventional LA method using a latex binding substance, an increase in background can be prevented.

Further, in the detection tool of the present invention, the channel also may serve as the sample holder (and further the reaction portion). In this case, for example, it is preferable that the detection reagent is placed in the channel. Further, at the time of using, when a liquid sample is supplied, passed through the channel, and then reached a placement portion of the detection reagent in the channel, the detection reagent is dissolved or dispersed into the liquid sample at the placement portion of the detection reagent and thereby formed an aggregate. For example, as described above, detection of the aggregate can be performed by irradiating the light to the placement portion of the reagent and the detection portion also serving as a reaction portion.

The material of the main body is preferably a material not affecting detection of the aggregate, although it is not particularly limited. As described above, when measurement is performed by an optical method, the sample placement portion (light irradiating portion) in the main body, that is, an irradiating region of light, a detection region of transmitted light, a detection region of scattered light, and the like preferably are formed of a light transmissive material. Alternatively, the whole main body may be formed of such material. Examples of such material include polymethylmethacrylate (PMMA), polydimethylsiloxane (PDMS), polyethylene terephthalate (PET), polystyrene (PS), and the like; a glass; and the like.

The size of the detection tool of the present invention is not limited at all. In detection and analysis of a target substance, a downsized device such as a so-called microchip, a micro reactor, a microarray, and the like has become the mainstream. The detection tool of the present invention may have the size similar to that of a common downsized device, for example. In a case of a common microchip, the size thereof is, for example, the overall length of about 20 to 200 mm, the overall width of about 20 to 150 mm, the overall thickness of about 0.1 to 10 mm, the channel depth of about 0.01 to 1 mm, the channel width of about 0.01 to 1 mm, and the channel length of about 10 to 200 mm. For example, an amount of a sample to be supplied to a microchip of the aforementioned size is preferably about 1 to 1000 μL. In a case where the channel, in which the detection reagent is placed, also serves as the sample placement portion and an axial direction of the channel is a light irradiating direction, the length of the channel (i.e., the cell length) is, for example, a micrometer order, specifically about 10 to 1000 μm, and preferably about 100 μm.

Further, in recent years, further downsizing is required with respect to a detection tool used for detection of a target substance or the like. Therefore, a so-called micro TAS (micro total analysis system: μTAS) is attracting attention. Generally, a micro TAS is composed of a tiny chip, in which a tiny channel having the width and the depth of several tens μm to several hundreds μm, a reaction portion, a sample holder (for example, also referred to as a light irradiating portion or detection portion), and the like are formed. The micro TAS is a system that realizes all processes, for example, required for detecting the target substance, in its tiny chip. As described above, the size of the detection tool of the present invention is not limited at all. However, as described above, unlike the conventional latex particle, the detection reagent of the present invention does not reduce measurement sensitivity by being absorbed to a substrate such as a chip. Therefore, the detection tool of the present invention is also useful as a micro TAS. In the case of the micro TAS, the size thereof is, for example, the overall length of about 20 to 200 mm, the overall width of about 20 to 150 mm, the overall thickness of about 0.1 to 10 mm, the channel depth of about 1 to 1000 μm, the channel width of about 1 to 1000 μm, and the channel length of about 10 to 200 mm. For example, an amount of a sample to be supplied thereto is preferably about 1 to 1000 μL. In a case where the channel, in which the detection reagent is placed, also serves as the sample holder and an axial direction of the channel is a light irradiating direction, the length of the channel (i.e., the cell length) is, for example, a micrometer order, specifically about 10 to 1000 μm, and preferably about 100 μm.

Further, as described above, in a case where scattered light is measured, the detection tool of the present invention further includes a light blocking portion for blocking the light, and the light blocking portion is disposed in the intermediate position of the light path of direct light transmitted through the sample. In this Embodiment, for example, the sample means a sample that is in contact with the detection reagent of the present invention or a mixture containing the same (for example, a mixed liquid). In other words, the sample means a reaction system.

According to the detection tool, for example, unlike the conventional scattered light measurement device, it is not required to move the light receiving portion to a wanted angle or to set plural light receiving portions to plural angles for receiving scattered light. Further, according to the detection tool, for example, positions of the light source, the light receiving portion, and the sample placement portion in the scattered light measurement device can be fixed. Since a positional relationship can be stabilized in this manner, for example, the reliability of measurement accuracy further can be increased. Further, since this can be achieved by merely providing the light blocking portion to the detection tool, for example, the detection tool can be applied to the existing light intensity measurement device.

The light blocking portion in the detection tool is not particularly limited, and is applicable as long as it is disposed in the intermediate position of the light path of direct light transmitted through a sample. Further, the light blocking portion has the configuration similar to that of the scattered light measurement device described above except that it is disposed in the detection tool. Moreover, the detection tool has the configuration similar to that of the sample holding tool used for the scattered light measurement device described above except that the detection reagent and the light blocking portion are provided.

The detection tool is applicable as long as it is provided with the aforementioned light blocking portion, and can be used in a light intensity measurement device such as the existing spectrophotometer, scattered light measurement device, and the like.

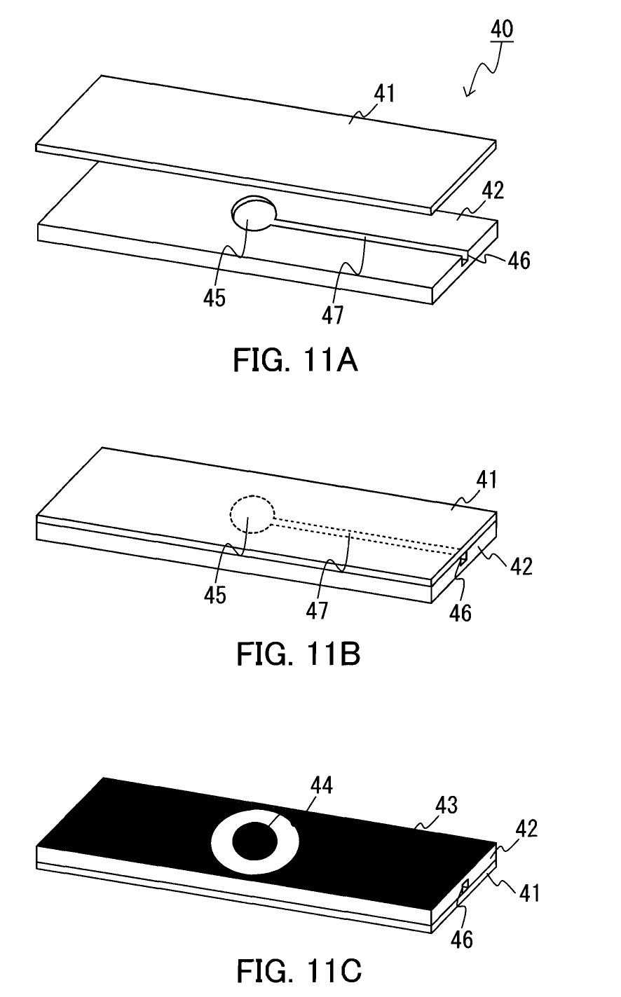

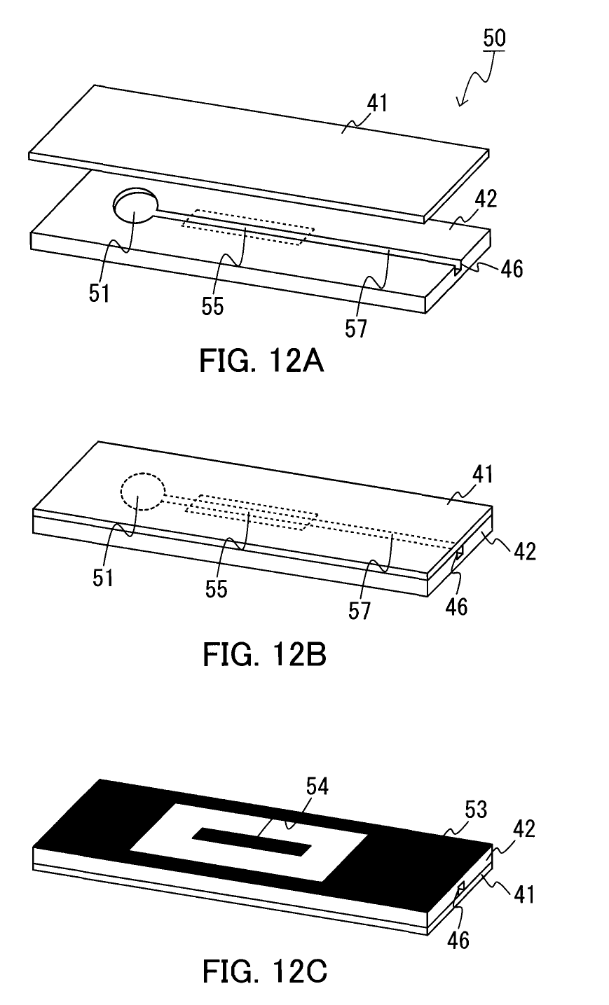

Hereinafter, specific examples of the detection tool provided with the light blocking portion are explained using FIGS. 11 and 12. In the detection tool of the following embodiment, a site where the detection reagent of the present invention is placed is not particularly limited, and is preferably the sample holder or a region (for example, channel) before the sample reaches the sample holder that is irradiated with the light. However, the present invention is not limited thereto.

### Embodiment 3

Embodiment 3 is an example of a detection tool including a sample holder whose planar shape is circular. In this Embodiment, the sample holder may also be referred to as a light irradiating portion or a detection portion. In a case where the detection reagent is placed in the sample holder, it may be referred to as a reaction portion (hereinafter, the same applies). The detection tool of this embodiment is not particularly limited as long as it includes the light blocking portion.

FIG. 11 is a perspective view of a detection tool 40, FIG. 11A is a schematic view in which each component is shown separately for convenience sake, FIG. 11B is a perspective view viewed from the upper side of the detection tool 40, and FIG. 11C is a perspective view viewed from the back side of the detection tool 40. As shown in FIG. 11, the detection tool 40 includes a support substrate 42 and a cover substrate 41. On the surface of the support substrate 42, a channel 47 and a sample holder 45 that is in communication with the channel 47 are formed as a concave groove. The support substrate 42 is covered with the cover substrate 41 at the surface where the concave groove is formed. Thereby, an end of the channel 47 (a right end in FIG. 11) serves as a sample introduction portion 46. As shown in FIG. 11C, a first light blocking portion 44 and a second light blocking portion 43 are formed at the back side of the detection tool 40. The first light blocking portion 44 is formed in the intermediate position of the light path of the direct light transmitted through the sample when the sample held at the sample holder 45 is irradiated with the irradiating light from the surface of the cover substrate 41 in the detection tool 40. Further, the second light blocking portion 43 is formed in the intermediate position of the light path of the scattered light transmitted through the sample and changed in path to an unwanted angle when the sample held at the sample holder 45 is irradiated with the irradiating light from the surface of the cover substrate 41 in the detection tool 40. However, the second light blocking portion 43 is optional and does not limit the present invention.

Types of the light to be irradiated to the detection tool 40 of this Embodiment are not particularly limited. For example, since the planar shape of the sample holder 45 is circular, it is preferable that the detection tool 40 is irradiated with the light emitted from the small-diameter light source. The size of the sample holder, conditions of the irradiating light from the small-diameter light source, and the like are not particularly limited and are as described above.

Using this detection tool 40, for example, the scattered light measurement method is performed as follows. First, a sample is supplied to the sample holder 45 from the introduction portion 46 of the detection tool 40. In a case where the detection reagent of the present invention is placed in the channel 47, for example, by passing through the sample, the detection reagent is dissolved or suspended into the sample and reaches the sample holder 45. Alternatively, in a case where the detection reagent of the present invention is placed at the sample holder 45, the sample reaches the sample holder 45 through the channel and the detection reagent then is dissolved or suspended into the sample. Further, the detection tool 40 in which the sample is held is disposed in a device that can receive light and measure its intensity, such as the existing spectrophotometer, scattered light measurement device, and the like. The configuration of such device is not particularly limited. For example, such device includes a light source emitting light, a light receiving portion receiving light, and a sample placement portion in which a sample is placed. The light receiving portion is disposed in a direction of light path of the irradiating light from the light source, and the sample placement portion is disposed between the light source and the light receiving portion. The detection tool 40 is disposed at the sample placement portion of such device and the sample at the sample holder 45 is irradiated with the irradiating light from the light source from the cover substrate 41 surface side of the detection tool 40. Then, the direct light transmitted through the sample out of the irradiating light to the sample is blocked at the first light blocking portion 44. Further, the scattered light, transmitted through the sample and changed in path to an unwanted angle, out of the irradiating light irradiated to the sample is blocked at the second light blocking portion 43. In contrast, the scattered light, transmitted through the sample and reaching a region between the first light blocking portion 44 and the second light blocking portion 43, out of the scattered light occurring due to contact with the sample is transmitted through the support substrate 42 of the detection tool 40 and received at the light receiving portion located below the detection tool 40. In this manner, the wanted scattered light efficiently can be received by blocking the direct light and further blocking the unwanted scattered light. The second light blocking portion 43 is optional and does not limit the present invention.

A method of supplying a sample into the detection tool 40 is not particularly limited. For example, the detection tool 40 further may include a component for introducing the sample. For example, the cover substrate 41 may include an air port above the sample holder 45. According to such Embodiment, for example, the sample may be introduced into the detection tool 40 from the sample introduction port 46 due to a capillary phenomenon. Alternatively, the sample may be introduced into the detection tool 40 due to pressure using a syringe or the like. Alternatively, the sample holder may be in communication with another channel, and the sample may be introduced into the detection tool 40 from the introduction port 46 by reducing the pressure inside of the detection tool 40 from an end opening of the channel. Alternatively, the sample may be introduced into the detection tool 40 from the introduction port 46 by aspirating the sample with a pump, a syringe, or the like from an end opening of the channel.

### Embodiment 4

Embodiment 4 is an example of a detection tool in which a channel is also serving as a sample holder. The sample holding tool of Embodiment 4 has the configuration similar to that of Embodiment 3 described above except that the channel also serves as the sample holder, unless otherwise described.

FIG. 12 is a perspective view of a detection tool 50, FIG. 12A is a schematic view in which each component is shown separately for convenience sake, FIG. 12B is a perspective view viewed from the upper side of the detection tool 50, and FIG. 12C is a perspective view viewed from the back side of the detection tool 50. As shown in FIG. 12, the detection tool 50 of Embodiment 4 may have the configuration similar to that of Embodiment 3 except that a part of a channel 57 also serves as a sample holder 55 that is irradiated with the light, and the planar shape of a first light blocking portion 54 and the shape of the seating rim of a second light blocking portion 53 are rectangular. Specifically, on the surface of the support substrate 42, the channel 57 and a waste liquid portion 51 that is in communication with the channel 57 are formed as a concave groove. As shown in FIG. 12C, the first light blocking portion 54 and the second light blocking portion 53 are formed at the back side of the detection tool 50. In the same manner as in Embodiment 3, the first light blocking portion 54 is formed in the intermediate position of the light path of the direct light transmitted through the sample and the second light blocking portion 53 is formed in the intermediate position of the light path of the scattered light transmitted through the sample and changed in path to an unwanted angle. However, the second light blocking portion 53 is optional and does not limit the present invention.

Types of the light to be irradiated to the detection tool 50 of this Embodiment are not particularly limited. For example, since the planar shape of the channel 57 (sample holder 55 irradiated with the light), in which the sample is held, is rectangular, it is preferable that the detection tool 50 is irradiated with the light emitted from the line light source. The size of the sample holder 55, conditions of the irradiating light from the line light source, and the like are not particularly limited and are as described above.

The detection tool of this Embodiment can be used in the same manner as the detection tool of Embodiment 3. Further, with respect to the sample introduced into the detection tool 50, the surplus thereof may be sent to the waste liquid portion 51.

Hereinafter, the present invention further is explained specifically with Examples. However, the present invention is not limited thereto.

Next, Examples of the present invention are explained with Comparative Examples. However, the present invention is not limited by the following Examples and Comparative Examples.

### EXAMPLE

### Example 1

CRP measurement was performed using an anti-CRP antibody in which various biotins and avidins are bound.

### <Production of Avidin-Biotinylated Anti-CRP Antibody)

First, biotin was bound to an anti-CRP antibody. 494.2 μL of a biotin solution (solvent: water) of 10 mmol/L was added to 3 mL of an anti-CRP antibody (manufactured by Oriental Yeast Co. Ltd.) of 12.2 mg/mL and the mixture thereof was left stationary at a room temperature. As the biotin, EZ-Link (trade mark) NHS-LC-LC-Biotin (manufactured by Pierce Biotechnology, Inc.) and EZ-Link (trade mark) TFP-PEG₃-Biotin (manufactured by Pierce Biotechnology, Inc.) were used respectively. The mixture was subjected to a centrifugal desalting column, Zeba (trade mark) Desalt Spin Columns (manufactured by Pierce Biotechnology, Inc.), and centrifuged under the condition of 1,000×g at 20° C. for 2 minutes, and thereby unreacted biotins were removed. Then, a solution containing the biotinylated anti-CRP antibody remaining in the column was collected and subjected to an ultrafiltration filter (molecular weight cut off of 100 k Da), centrifuged under the condition of 3,600×rpm at 20° C. for 20 minutes, and thereby the biotinylated anti-CRP antibody was concentrated. In this state, a biotin labeling ratio of the anti-CRP antibody was decided by a HABA substitution method. As a result, it was confirmed that more than one molecule of biotins are bound per molecule of antibody. Further, in advance of avidination that is the next process, an amount of protein was measured with respect to a concentrated solution containing the biotinylated anti-CRP antibody. Measurement of the amount of protein was performed by an absorbance measurement at 280 nm. Subsequently, avidin further was bound to the biotinylated anti-CRP antibody. 1.35 mL of a PBS solution containing avidin (manufactured by Calbiochem) of 2 mg/mL was added to the aforementioned concentrated solution containing the biotinylated anti-CRP antibody, and the mixture thereof was left stationary at a room temperature for 5 minutes. Subsequently, the mixture was subjected to an ultrafiltration filter (molecular weight cut off of 100 k Da), centrifuged under the condition of 3,600×rpm at 20° C. for 20 minutes, and thereby unreacted avidins were removed and an avidin-biotinylated anti-CRP antibody was concentrated. In this manner, the avidin-biotinylated anti-CRP antibody was obtained. In this state, the avidin-biotinylated anti-CRP antibody is referred to as an avidin-NHS-LC-LC-biotinylated anti-CRP antibody when EZ-Link (trade mark) NHS-LC-LC-Biotin was used as biotin, and to as an avidin-TFP-PEG₃-biotinylated anti-CRP antibody when EZ-Link (trade mark) TFP-PEG₃-Biotin was used as biotin.

### <Preparation of Dried Reagent>

Using the avidin-biotinylated anti-CRP antibody, liquid reagents of the following composition were prepared. In the following composition, an amount of the avidin-NHS-LC-LC-biotinylated anti-CRP antibody to be added was 29.3 μL and an amount of the avidin-TFP-PEG₃-biotinylated anti-CRP antibody to be added was 26.4 μL. 29 μL of liquid regents thereof were introduced into a cell having the cell length of 100 μm and dried by freeze dry, respectively. In this manner, cells in which each dried reagent is placed were produced respectively.

TABLE 3
(Liquid Reagent Composition; pH 7.2)

| Avidin-biotinylated anti-CRP antibody | 0.62 | mg (protein amount) |
|---|---|---|
| 50% PEG 6000 | 0.85 | μL |
| 10% NaN₃ | 0.023 | μL |
| TritonX-305 (manufactured by | 0.023 | μL |
| Nacalai Tesque, Inc.) | | |
| 2.5 M MOPS buffer solution | 1.7 | μL |
| (manufactured by Dojindo Laboratories) | | |
| Purified water | Predetermined amount | |
| Total | 29 | μL |

### <CRP Concentration Measurement Method>

CRP (manufactured by Capricorn Products LLC.) was added to CRP-free blood serum (manufactured by Oriental Yeast Co. Ltd.) collected from human whole blood so as to have a predetermined concentration, and thereby a specimen was prepared. The predetermined concentration was 0, 1.2, 5, 6, 10, 20, 42.9, 65, and 78 mg/100 mL. Then, 20 μL of each specimen was added to the aforementioned cells each having dried reagent of each avidin-biotinylated anti-CRP antibody, and incubated at 25° C. for 4 minutes. Thereafter, transmitted light was measured at the wave length ranges of 405 nm and 810 nm using a spectrophotometer. Intensity of the transmitted light at 810 nm was subtracted from intensity of the transmitted light at 405 nm to calculate an absorbance (Abs. (405-810 nm)).

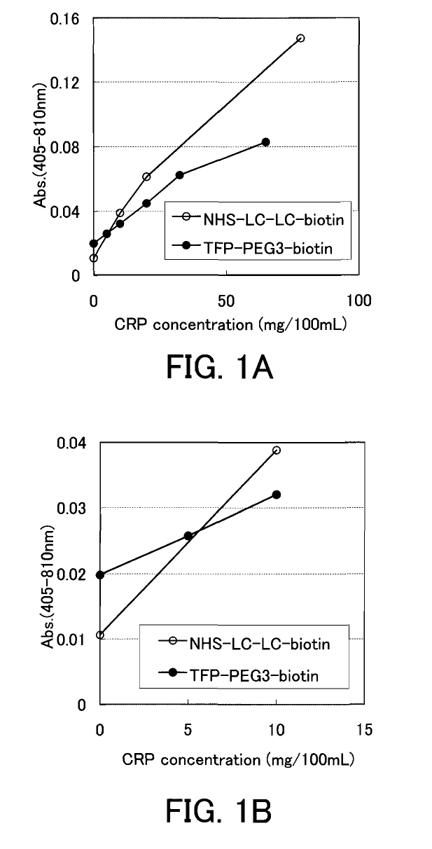

Results thereof are shown in FIGS. 1A and 1B. FIG. 1A is a graph that shows an absorbance in each CRP concentration, and FIG. 1B is a graph in which a range of CRP concentration of 0 to 10 mg/100 mL is enlarged. In FIG. 1, a white circle shows results of a case using NHS-LC-LC-biotin as biotin and a black circle shows results of a case using TFP-PEG₃-biotin as biotin.

As shown in FIG. 1A, in a range of CRP concentration of 10 to 78 mg/100 mL, in both cases of using NHS-LC-LC-biotin and TFP-PEG₃-biotin as biotin, an absorbance was increased approximately in proportion to a CRP concentration. Also, in a high concentration range, prozone was not found. Further, as shown in FIG. 1B, in a range of CRP concentration of 0 to 10 mg/100 mL, in both cases of using NHS-LC-LC-biotin and TFP-PEG₃-biotin as biotin, an absorbance was increased approximately in proportion to a CRP concentration. Also, in both cases of using NHS-LC-LC-biotin and TFP-PEG₃-biotin as biotin, the background showed a sufficiently low value such as about 0.01 to 0.02.

According to the above results, it was found that CRP of about 0 to 78 mg/100 mL could be measured quantitatively, without diluting a sample, with the cell length of micrometer order by using the avidin-biotinylated anti-CRP antibody.

### Example 2

CRP measurement was performed by changing the ratio between antigen CRP and an avidin-biotinylated anti-CRP antibody.

<Preparation of Dried Reagent containing Avidin-Biotinylated Anti-CRP Antibody>

Each dried reagent respectively was placed in cells in the same manner as in Example 1 except that 29.3 μL of avidin-NHS-LC-LC-biotinylated anti-CRP antibody was used.

### <CRP Concentration Measurement Method>

In the same manner as in Example 1, specimens each containing antigen CRP of predetermined concentration were prepared. The predetermined concentration was 0, 10, 20, and 78 mg/100 mL. Each specimen was added to the aforementioned cells, and then an absorbance was measured in the same manner as in Example 1. An amount of each specimen to be added was 15, 20, 40, 50 and 60 μL.

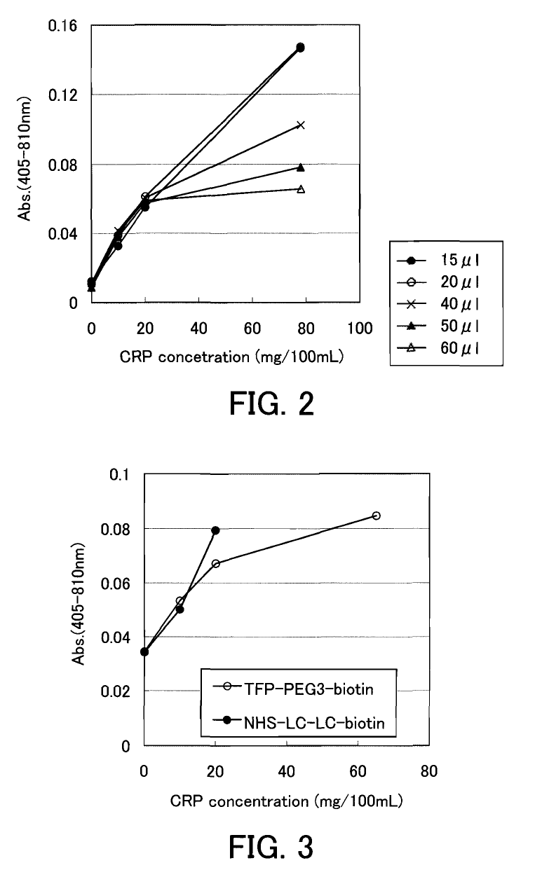

Results thereof are shown in FIG. 2. FIG. 2 is a graph that shows an absorbance in each CRP concentration. The plot in FIG. 2 shows results of each specimen, and a black circle shows a result of 15 μL, a white circle shows a result of 20 μL, an X shows a result of 40 μL, a black triangle shows a result of 50 μL, and a white triangle shows a result of 60 μL.

As shown in FIG. 2, in a case where an amount of the specimen to be added was 60 μL (white triangle), although an increase in an absorbance in proportion to a CRP concentration was confirmed in a low concentration range of 0 to 20 mg/100 mL, prozone was confirmed in a high concentration range of 20 mg to 78 mg/100 mL. However, as shown in a case of 50 μL (black triangle), 40 μL (X), 20 μL (white circle), and 15 μL (black circle), in accordance with reduction of an amount of the specimen to be added, i.e., in accordance with increase in an antibody ratio relative to antigen, prozone was resolved. In particular, as shown in the black circle of FIG. 2, it was found that an absorbance was increased in proportion to a CRP concentration in a range of 0 to 78 mg/100 mL, i.e., a whole range from a low concentration range to a high concentration range, by reducing an amount of the specimen to be added to 15 μL. Further, the background showed a sufficiently low value such as about 0.01 regardless of an amount of the specimen to be added.

According to the above results, for example, even in a case where prozone occurred in a high concentration range, it was found that use of an avidin-biotinylated anti-CRP antibody made it possible effectively to increase the measurement accuracy by adjusting an antibody ratio relative to antigen. Further, regardless of the occurrence of prozone, also in a low concentration range, measurement could be performed in excellent sensitivity and the background showed a sufficiently low value. Therefore, it was found that excellent measurement sensitivity and measurement accuracy could be achieved. According to the avidin-biotinylated antibody of this example, it was found that CRP of about 0 to 78 mg/100 mL could be measured quantitatively, without diluting a sample, with the cell length of micrometer order.

### Example 3

CRP measurement was performed using a streptavidin-biotinylated anti-CRP antibody, in which streptavidin was added instead of avidin.

### <Production of Streptavidin-Biotinylated Anti-CRP Antibody>

A streptavidin-biotinylated anti-CRP antibody was produced in the same manner as in Example 1 except that streptavidin (manufactured by Pierce Biotechnology, Inc.) was used instead of biotin. In this state, the streptavidin-biotinylated anti-CRP antibody is referred to as a streptavidin-NHS-LC-LC-biotinylated anti-CRP antibody when EZ-Link (trade mark) NHS-LC-LC-Biotin was used as biotin, and to as a streptavidin-TFP-PEG₃-biotinylated anti-CRP antibody when EZ-Link (trade mark) TFP-PEG₃-Biotin was used as biotin.

### <Preparation of Dried Reagent>

Liquid reagents were prepared and dried reagents were placed in cells in the same manner as in Example 1 except that 25.2 μL of streptavidin-biotinylated anti-CRP antibody was added instead of avidin-biotinylated anti-CRP antibody.

### <CRP Concentration Measurement Method>

In the same manner as in Example 1, specimens each containing antigen CRP of predetermined concentration (0, 10, 20, and 78 mg/100 mL) were prepared. Each specimen was added to the aforementioned cells, and then an absorbance was measured in the same manner as in Example 1. An amount of the specimen to be added was 35 μL with respect to the cell containing the streptavidin-NHS-LC-LC-biotinylated anti-CRP antibody, and was 29.4 μL with respect to the cell containing the streptavidin-TFP-PEG₃-biotinylated anti-CRP antibody.

Results thereof are shown in FIG. 3. FIG. 3 is a graph that shows an absorbance in each CRP concentration. In FIG. 3, a white circle shows the results of a case using the streptavidin-TFP-PEG₃-biotinylated anti-CRP antibody and a black circle shows the results of a case using the streptavidin-NHS-LC-LC-biotinylated anti-CRP antibody.

As shown in FIG. 3, in a case of using the streptavidin-TFP-PEG₃-biotinylated anti-CRP antibody, in a range of CRP concentration of 0 to 65 mg/100 mL, an absorbance was increased approximately in proportion to a CRP concentration. Further, as shown in FIG. 3, also in a case of using the streptavidin-NHS-LC-LC-biotinylated anti-CRP antibody, in a range of CRP concentration of 0 to 20 mg/100 mL, an absorbance was increased approximately in proportion to a CRP concentration. Moreover, in both cases of using the streptavidin-TFP-PEG₃-biotinylated anti-CRP antibody and the streptavidin-NHS-LC-LC-biotinylated anti-CRP antibody, the background showed a sufficiently low value such as 0.04 or less.

According to the above results, it was found that CRP could quantitatively be measured with low background values also in a case of using streptavidin. According to the streptavidin-biotinylated antibody of this Example, it was found that CRP of about 0 to 78 mg/100 mL could quantitatively be measured, without diluting a sample, with the cell length of micrometer order.

### Example 4

CRP concentration measurement was performed using a biotinylated anti-CRP antibody in which only biotins are bound.

### <Production of Biotinylated Anti-CRP Antibody>

A biotinylated anti-CRP antibody was produced in the same manner as in Example 1 except that avidins were not bound. As the biotin, EZ-Link (trade mark) NHS-LC-LC-Biotin (manufactured by Pierce Biotechnology, Inc.) was used.

### <Preparation of Dried Reagent>

A liquid reagent was prepared and a dried reagent was placed in a cell in the same manner as in Example 1 except that 19.2 μL of biotinylated anti-CRP antibody was added instead of avidin-biotinylated anti-CRP antibody.

### <CRP Concentration Measurement Method>

In the same manner as in Example 1, specimens each containing antigen CRP of predetermined concentration (0, 1.2, 3, 6, 10, 20, 42.9, and 78 mg/100 mL) were prepared. Each specimen was added to the aforementioned cell, and then an absorbance was measured in the same manner as in Example 1. An amount of the specimen to be added was 25 μL.

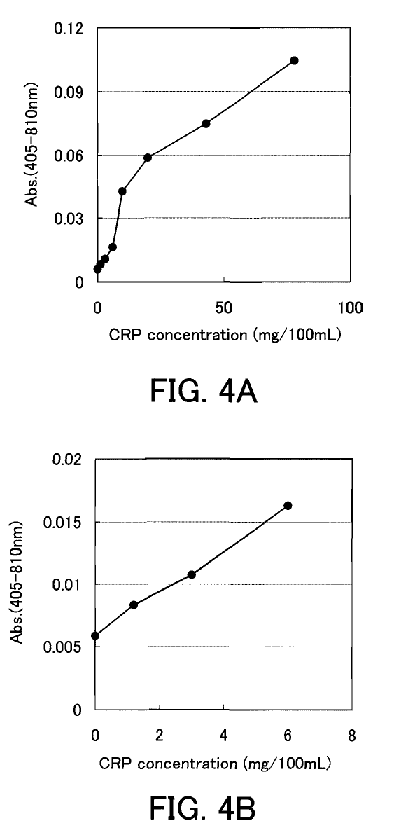

Results thereof are shown in FIGS. 4A and 4B. FIG. 4A is a graph that shows an absorbance in each CRP concentration, and FIG. 4B is a graph in which a range of CRP concentration of 0 to 6 mg/100 mL is enlarged.

As shown in FIG. 4A, in a range of CRP concentration of 0 to 78 mg/100 mL, an absorbance was increased approximately in proportion to a CRP concentration. In particular, as shown in FIG. 4B, even in a low concentration range (CRP of 0 to 6 mg/100 mL) in which measurement was hardly performed with the conventional TIA method, an absorbance was increased approximately in proportion to a CRP concentration. Further, the background showed a sufficiently low value such as 0.01 or less.

According to the above results, also in a case of using the biotinylated anti-CRP antibody in which only biotins are bound, it was found that CRP could be measured, without receiving a disadvantageous effect due to prozone, with low background. According to the biotinylated antibody of this Example, it was found that CRP of about 0 to 78 mg/100 mL could be measured quantitatively, without diluting a sample, with the cell length of micrometer order.

### Example 5

Background was measured using an avidin-biotinylated anti-CRP antibody and a streptavidin-biotinylated anti-CRP antibody.

### <Production of Avidin-Biotinylated Anti-CRP Antibody and Streptavidin-Biotinylated Anti-CRP Antibody)

An avidin-biotinylated anti-CRP antibody and a streptavidin-biotinylated anti-CRP antibody were produced in the same manner as in Examples 1 and 3 using biotin (EZ-Link (trade mark) NHS-LC-LC-Biotin) and avidin or streptavidin.

### <Preparation of Dried Reagent>

Liquid reagents were prepared and dried reagents were placed in cells in the same manner as in Example 1 except that 27.8 μL of avidin-biotinylated anti-CRP antibody or 29.4 μL of streptavidin-biotinylated anti-CRP antibody was added.

### <Background Measurement Method>

20 μL of CRP-free blood serum (manufactured by Oriental Yeast Co. Ltd.) collected from human whole blood was added to the aforementioned cells, and an absorbance was calculated in the same manner as in Example 1.

As a result, the background in a case of using the streptavidin-biotinylated anti-CRP antibody was 0.035 and the background in a case of using the avidin-biotinylated anti-CRP antibody was 0.019. In this manner, in both cases of using the streptavidin-biotinylated anti-CRP antibody and the avidin-biotinylated anti-CRP antibody, the background showed a sufficiently low value. Particularly, it was found that use of avidin made it possible to further reduction of the background.

### Example 6

CRP was measured by detecting transmitted light and scattered light.

A dried reagent was placed in a cell in the same manner as in Example 1 using the avidin-NHS-LC-LC-biotinylated anti-CRP antibody prepared in Example 1 as the avidin-biotinylated anti-CRP antibody. In this state, an amount of the avidin-biotinylated anti-CRP antibody to be added was 18 μL.

### <CRP Concentration Measurement Method>

In the same manner as in Example 1, specimens each containing antigen CRP of predetermined concentration (0, 0.1, 0.5, 1, 5, 10, 20, 32.5, and 65 mg/100 mL) were prepared. Then, 20 μL of each specimen was added to the aforementioned cell. Thereafter, the intensity of transmitted light was measured at the wave length ranges of 405 nm and 810 nm in the same manner as in Example 1 and an absorbance was calculated by subtracting the intensity of the transmitted light at 810 nm from the intensity of the transmitted light at 405 nm.

On the other hand, 40 μL of each specimen was added to the sample holding tool (cell) described below and incubated at 25° C. for 4 minutes. Thereafter, the intensity of the scattered light (at 365 nm) was measured using the scattered light measurement device described below.

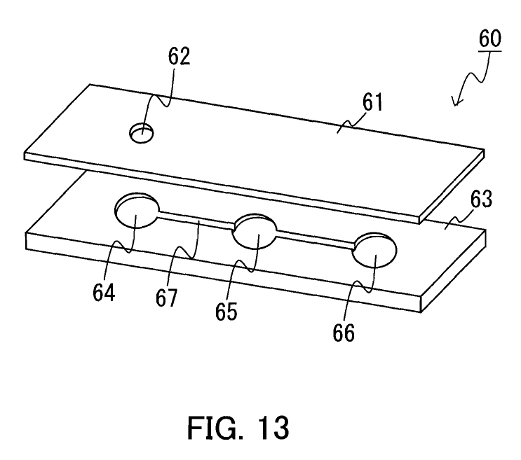

29 μL of the liquid reagent same as Example 1 was subjected to a sample holder 65 of a sample holding tool 60 shown in FIG. 13 and was dried by freeze drying. In this manner, a dried reagent was placed in the sample holding tool 60. FIG. 13 is a perspective view showing an example of a sample holding tool and is a schematic view in which each component is shown separately for convenience sake. As shown in FIG. 13, the sample holding tool 60 includes a support substrate 63 and a cover substrate 61. On the surface of the support substrate 63, a sample introduction portion 64, the sample holder 65 in which the dried reagent is placed, a waste liquid portion 66, and a channel 67 are formed as a concave groove. The sample introduction portion 64, the sample holder 65, and the waste liquid portion 66 are in communication among one another through the channel 67. The cover substrate 61 is provided with a sample introduction port 62 at the site corresponding to the sample introduction portion 64 of the support substrate 63. The support substrate 63 is covered with the cover substrate 61 at the surface where the groove is formed. In the sample holding tool 60, the planar shape of the sample holder 65, i.e., the shape of the surface that is irradiated with light, is circular. Details of the sample holding tool 60 are as follows:

### Cover Substrate

Material: Silicon polyethylene terephthalate (PET)
Thickness: 150 μm

### Support Substrate

Material: Acrylic resin
Thickness: 5 mm (length from bottom surface of sample holder to bottom surface of support substrate)

### Sample Holder

Diameter: 1.3 mm
Depth: about 300 μm

### <Scattered Light Measurement Device>

The scattered light measurement device shown in FIG. 8 was assembled. Details of each component of the scattered light measurement device are as follows. In this Example, a sample holding tool 12 in FIG. 8 is the sample holding tool 60 of FIG. 13 described above.
Light Source: LED (manufactured by NITRIDE SEMICONDUCTORS. CO., Ltd.)
Wave Length: 365 nm
Irradiating Light Diameter: 0.4 mm
Light Receiving Portion: Photodiode (trade name: S2386-44K, manufactured by Hamamatsu Photonics K. K.)

### First Light Blocking Portion

Material: Acrylic containing 50% (w/t) of shading agent of carbon black
Diameter: 0.8 mm
Thickness: 10 μm

### Second Light Blocking Portion

Material: Acrylic containing 50% (w/t) of shading agent of carbon black
Inner diameter: 25 mm
Thickness: 10 μm

20 μL of each specimen was added to the sample holding tool 60 from the introduction port 62, transferred from the sample introduction portion 64 to the sample holder 65, and incubated at 25° C. for 4 minutes. Thereafter, using the scattered light measurement device, the intensity of the scattered light (at 365 nm) was measured by irradiating light on the sample holder 65. In the scattered light measurement device shown in FIG. 8, the sample holding tool 60 is placed such that the sample holder 65 is irradiated with light, and direct light transmitted through the sample is blocked at the first light blocking portion.

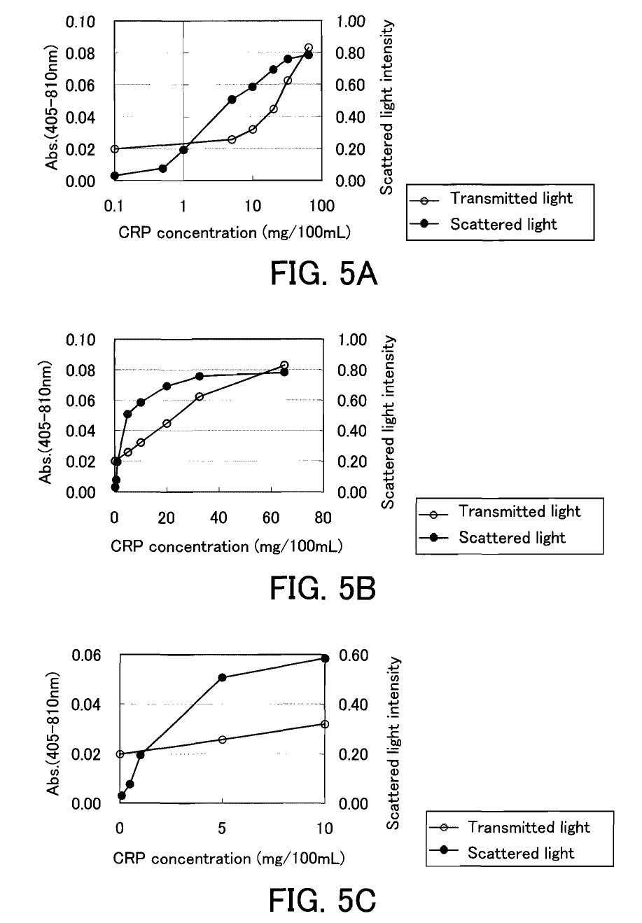

Results thereof are shown in FIGS. 5A to 5C. FIG. 5A is a graph that shows a relationship between a logarithmic displayed CRP concentration and an absorbance as well as a relationship between a logarithmic displayed CRP concentration and a scattered light intensity. FIG. 5B is a graph in which the CRP concentration shown in FIG. 5A is displayed as constant. FIG. 5C is a graph in which the CRP concentration of 0 to 10 mg/100 mL in FIG. 5B is enlarged. In FIG. 5, a white circle shows results of the absorbance and a black circle shows the results of the scattered light intensity.

As shown in the white circle (absorbance) of FIGS. 5A to 5C, with respect to the measurement of the transmitted light, in both ranges of low concentration and high concentration, an absorbance was increased in proportion to a CRP concentration. Further, the background showed a sufficiently low value such as about 0.02. In contrast, with respect to the measurement of the scattered light, the scattered light intensity was increased in proportion to a CRP concentration as shown in the black circle (scattered light intensity) of FIG. 5A. As shown in FIG. 5C, measurement sensitivity in a low concentration range was increased further and the background showed a further low value such as 0.001 or less. As described above, according to this Examination, even with respect to the same reaction solution, detection of CRP could be performed by both of the turbidimetry that measures transmitted light and the nephelometry that measures scattered light. Further, it was found that CRP measurement could be performed with further excellent accuracy and sensitivity by measuring the scattered light.

### [Comparative Examination 1]

CRP measurement was performed using anti-CRP antibody by the conventional technology of the turbidimetric immunoassay (TIA) method.

### <Preparation of Dried Reagent>

A liquid reagent was prepared and a dried reagent was placed in a cell in the same manner as in Example 1 except that 19.2 μL of anti-CRP antibody was added instead of avidin-biotinylated anti-CRP antibody.

### <CRP Concentration Measurement Method>

In the same manner as in Example 1, specimens each containing antigen CRP of predetermined concentration (0, 1.2, 3, 6, 10, 20, 42.9, and 78 mg/100 mL) were prepared. Then, the predetermined amount (50, 75, 100 μL) of each specimen was added to the aforementioned cell, and then an absorbance was measured.

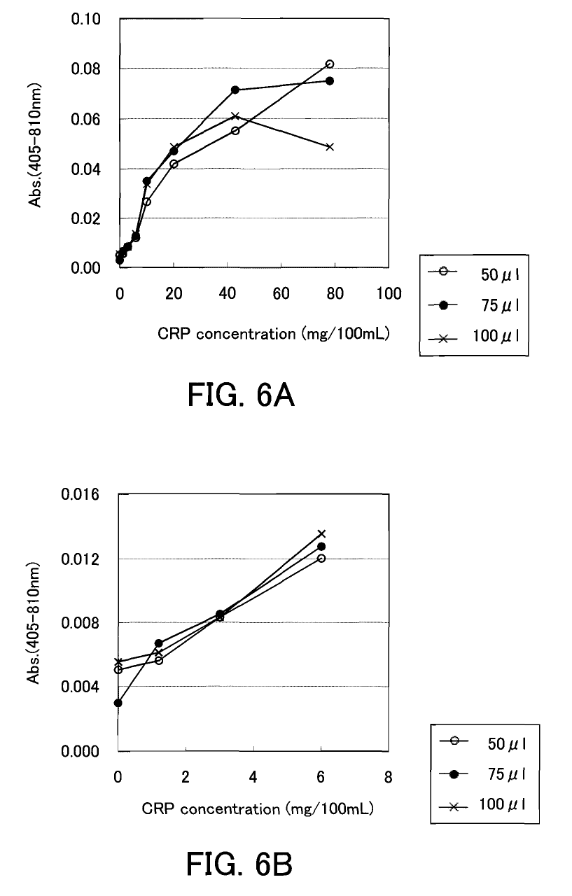

Results thereof are shown in FIGS. 6A and 6B. FIG. 6A is a graph that shows an absorbance in each CRP concentration. FIG. 6B is a graph in which a range of CRP concentration of 0 to 6 mg/100 mL is enlarged. In FIG. 6, a white circle shows results of a case of 50 μL, a black circle shows results of a case of 75 μL, and an X shows results of a case of 100 μL, respectively.

As shown in FIG. 6A, in a case of adding 100 μL of specimen (X), prozone was confirmed in a CRP high concentration range. The occurrence of prozone was resolved in accordance with reduction of an amount of the specimen to be added such as to 75 μL (black circle) then to 50 μL (white circle), i.e., in accordance with the increase in an antibody ratio relative to antigen. However, with respect to the TIA method, problems also were found in a low concentration range. That is, as shown in FIG. 6B, in a CRP low concentration range, although an absorbance was increased in proportion to a concentration regardless of an amount of the specimen to be added, there was little difference with background and the absorbance was very low. Therefore, it was found that the measurement sensitivity in a low concentration range was insufficient. Comparing Comparative Example 1 and each Example, according to Examples using biotinylated antibody or avidin-biotinylated antibody, because excellent sensitivity also was shown in a low concentration range, it was found that measurement could be performed with respect to a wide concentration range and the occurrence of prozone could effectively be restrained.

### Comparative Example 2

CRP measurement was performed using anti-CRP antibody by the conventional technology of the latex agglutination-turbidimetric immunoassay (LA) method.

22.5 μL of reagent R1 and 112.5 μL of reagent R2 of of a commercially available reagent (trade name: Iatro CRP-EX, manufactured by Mitsubishi Kagaku latron, Inc.) containing a latex binding anti-CRP antibody were introduced in separate containers and dried by freeze drying. On the other hand, in the same manner as in Example 1, specimens each containing antigen CRP of predetermined concentration (0, 0.5, 0.85, 3, 6, 10, 20, 42.9, and 78 mg/100 mL) were prepared. Then, 9 μL of each specimen was added to the dried R1 to dissolve R1 into the specimen, and left for 2 minutes. Thereafter, the dried R2 was dissolved with the total amount of this reaction solution. Then, 2 minutes after dissolution of R2, measurement of the transmitted light and calculation of the absorbance were performed in the same manner as in Example 1.

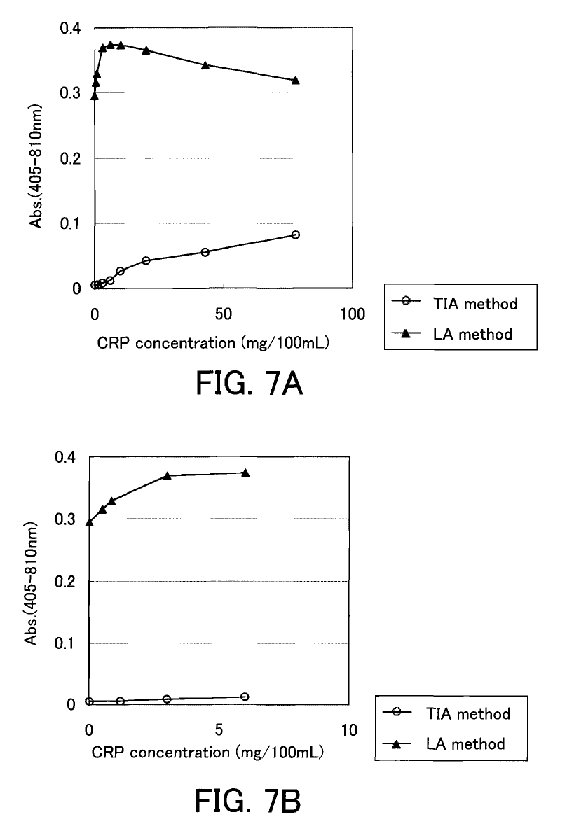

Results thereof and a result of a case of adding 100 μL of specimen in Comparative Example 1 are shown in FIG. 7. FIG. 7A is a graph that shows an absorbance in each CRP concentration, and FIG. 7B is a graph in which a range of CRP concentration of 0 to 6 mg/100 mL in FIG. 7A is enlarged. In FIG. 7, a black triangle shows results of the LA method in Comparative Example 2 and a white circle shows results of the TIA method in Comparative Example 1.

As shown in FIG. 7A, in a case of the LA method (black triangle), although an absorbance was increased in proportion to a CRP concentration, prozone was confirmed in a very low CRP concentration range such as 3 mg/100 mL. This prozone could not be improved by changing the antigen-antibody ratio and this was the limit data obtained with respect to an undiluted sample. Further, as shown in FIG. 7B, the background showed a very high value such as Abs. 0.29. From this result, with respect to the LA method, as compared to the TIA method, although measurement in a low concentration range could be performed, it was found that accuracy and sensitivity in a wide concentration range were insufficient because there are problems of background and notable prozone. Further, it was found that prozone could not be resolved without diluting the sample. Comparing Comparative Example 2 and each Example, according to Examples using biotinylated antibody or avidin-biotinylated antibody, because excellent sensitivity was also shown in a low concentration range, it was found that measurement could be performed with respect to a wide concentration range, the background could sufficiently be reduced, and occurrence of prozone could effectively be restrained. Therefore, according to each Example, CRP in a wide concentration range could quantitatively be measured, without diluting a sample, with the cell length of micrometer order.

As described above, according to the present invention, a target substance can be detected with excellent accuracy and sensitivity by using the aforementioned modified binding substance. Specifically, according to the method of the present invention, for example, measurement in a low concentration range, which could hardly be measured with the TIA method, and measurement in a high concentration range, which hardly could be measured with the LA method due to the occurrence of prozone, can be performed. Accordingly, the present invention makes it possible to detect a target substance with excellent accuracy and sensitivity also in a concentration range where measurement could not be performed by the conventional methods. Therefore, the present invention is a very useful new method in an analysis and clinical field.

The invention may be embodied in other forms without departing from the spirit or essential characteristics thereof. The embodiments disclosed in this application are to be considered in all respects as illustrative and not limiting. The scope of the invention is indicated by the appended claims rather than by the foregoing description, and all changes which come within the meaning and range of equivalency of the claims are intended to be embraced therein.

1. A detection method of detecting a target substance in a sample using a binding substance that binds to the target substance, comprising the following (A) and (B):
(A) bringing the binding substance into contact with the sample, and forming an aggregate by binding of the binding substance and the target substance in the sample; and
(B) detecting the aggregate, wherein
the binding substance is a binding substance to which modifying substances having a maximum diameter of about 50 nm or less bind.

2. The detection method according to claim 1, wherein the modifying substance includes at least one of biotin and a biotin derivative.

3. The detection method according to claim 2, wherein the modifying substance further includes at least one of avidin and an avidin derivative, and at least one of the avidin and the avidin derivative is bound to at least one of the biotin and the biotin derivative.

4. The detection method according to claim 3, wherein the avidin derivative is at least one selected from the group consisting of streptavidin, modified streptavidin, and deglycosylated avidin.

5. The detection method according to claim 1, wherein the modifying substance further includes a spacer, and at least one of the biotin and the biotin derivative is bound to the binding substance via the spacer.

6. The detection method according to claim 1, wherein the target substance is an antigen and the binding substance is an antibody, or the target substance is an antibody and the binding substance is an antigen.

7. The detection method according to claim 1, wherein in the (A), the binding substance to which the modifying substances bind is in a dry condition before being brought into contact with the sample.

8. The detection method according to claim 1, wherein the sample is an undiluted sample.

9. The detection method according to claim 1, wherein the sample is blood.

10. The detection method according to claim 1, wherein the target substance is C-reactive protein.

11. The detection method according to claim 1, wherein in the (B), the aggregate is detected by detection of at least one of scattered light and transmitted light.

12. A detection reagent used for a detection method of a target substance according to claim 1, comprising:
a binding substance that binds to the target substance, wherein
the binding substance is a binding substance to which modifying substances having the maximum diameter of about 50 nm or less bind.

13. The detection reagent according to claim 12, wherein the modifying substance includes at least one of biotin and a biotin derivative.

14. The detection reagent according to claim 13, wherein the modifying substance further includes at least one of avidin and an avidin derivative, and at least one of the avidin and the avidin derivative binds to at least one of the biotin and the biotin derivative.

15. The detection reagent according to claim 12, wherein the target substance is an antigen and the binding substance is an antibody, or the target substance is an antibody and the binding substance is an antigen.

16. A detection tool used for a detection method of a target substance according to claim 1, comprising:
a main body and a detection reagent comprising:
a binding substance that binds to the target substance, wherein the binding substance is a binding substance to which modifying substances having the maximum diameter of about 50 nm or less bind,
wherein the detection reagent is placed in the main body.

17. The detection tool according to claim 16, wherein the detection tool is a detection tool for detecting at least one of scattered light and transmitted light, and a cell length is about 10 to 1000 μm.

18. The detection tool according to claim 16, wherein the detection tool is a micro TAS.
